(19) United States
(12) Patent Application Publication
(10) Pub. No.: US 2007/0072175 A1
(43) Pub. Date: Mar. 29, 2007

(54) Nucleotide array containing polynucleotide probes complementary to, or fragments of, cynomolgus monkey genes and the use thereof

(75) Inventors: Matthew T. Cooper, Palo Alto, CA (US); Deborah Kinch, Boston, MA (US); Michael Rosenberg, Issaquah, WA (US); S. Sai Subramaniam, Sharon, MA (US); Suzanne T. Szak, Arlington, MA (US); Huo Li, Winchester, MA (US); Raj Bandaru, Malden, MA (US); Maher Derbel, Arlington, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 11/433,832

(22) Filed: May 15, 2006

Related U.S. Application Data
(60) Provisional application No. 60/680,473, filed on May 13, 2005.
(60) Provisional application No. 60/680,544, filed on May 13, 2005.

(51) Int. Cl. C12Q 1/70; C12Q 1/68; C12M 3/00

(52) U.S. Cl. 435/5; 435/6; 435/287.2

(57) ABSTRACT

This invention relates to a nucleotide array containing polynucleotide probes complementary to, or fragments of, Cynomolgus monkey genes, and the use of such a nucleotide array to characterize the biological effects, including the actions, targets, and toxicities, of therapeutic agents in primates, e.g., a human, a Cynomolgus monkey, or a Rhesus monkey, in particular a nucleotide array to be used in identifying the toxicities of therapeutic agents administered to a non-human primate, e.g., a Cynomolgus monkey or a Rhesus monkey.

### CROSS-REFERENCE TO RELATED APPLICATION

This application claims the benefit of U.S. Provisional Patent Application Nos. 60/680,473, filed May 13, 2005, and 60/680,544, filed May 13, 2005, herein incorporated by reference.

### REFERENCE TO A SEQUENCE LISTING AND TABLES SUBMITTED ON A COMPACT DISC

This application includes a “Sequence Listing” and Table 2 which are provided as electronic documents on a compact disk (CD-R). This compact disk contains the files “Sequence_Listing.txt” (59,586,560 bytes, created on May 15, 2006) and “Table 2.txt” (2,095,104 bytes, created on May 15, 2006), which are hereby incorporated by reference in their entirety.

### BACKGROUND OF THE INVENTION

1. Field of the Invention

A device and/or method that may be used to characterize the biological effects, including the actions, targets, and toxicities, of a therapeutic agent on a primate, e.g., a human, a Cynomolgus monkey or a Rhesus monkey, especially a non-human primate such as a Cynomolgus monkey or a Rhesus monkey, quickly and accurately would be useful in identifying which therapeutic agents warrant further development.

This invention relates to a nucleotide array containing polynucleotide probes complementary to, or fragments of, any portion of a Cynomolgus monkey gene, and the use of such a nucleotide array, to characterize the actions, targets, and toxicities of therapeutic agents in primates, e.g., a human, a Cynomolgus monkey or a Rhesus monkey, in particular a nucleotide array to be used in identifying the toxicities of therapeutic agents in a non-human primate such as a Cynomolgus or a Rhesus monkey.

2. Background

Drug discovery, a process by which bioactive compounds are identified and preliminarily characterized, is a critical step in the development of treatments for human diseases. Knowledge of all the primary targets of a therapeutic agent is necessary in understanding efficacy, side-effects, toxicities, possible failures of efficacy, and activation of metabolic responses. Further, the identification of all primary targets of a drug can lead to discovery of alternative primary targets suitable to achieve the original therapeutic response.

One phase of the drug discovery process involves utilizing animal studies to determine the toxicity of a therapeutic agent, such as in studies conducted in non-human primates, e.g., Cynomolgus or Rhesus monkeys. Toxicity analysis of therapeutic agents is often the rate-limiting step in the development of new pharmaceutical compounds. J. F. Waring et al. Toxicology Letters 120:359-368 (2001). Therefore, characterizing the effects of a therapeutic agent on the cellular metabolism of a non-human primate, e.g., a Cynomolgus or a Rhesus monkey, quickly and accurately would be useful in identifying which therapeutic agents warrant further development.

Microfabricated arrays of large numbers of polynucleotide probes, called “nucleotide arrays,” “DNA chips,” or “gene chips,” may be used to identify the primary targets of a therapeutic agent.

Currently available nucleotide arrays are based upon bovine, canine, human, mouse, and rat gene sequences. However, there is not an available nucleotide array based upon a non-human primate, e.g., a Cynomolgus or a Rhesus monkey, gene sequences that may be utilized in investigating the biological effects, including the actions, targets, and toxicities, of a therapeutic agent in primates, e.g., a human, a Cynomolgus monkey, or a Rhesus monkey.

Nucleotide arrays are used to detect complementary nucleic acid sequences in a nucleic acid of interest. In some assay formats, the polynucleotide probe is tethered, i.e., by covalent attachment, to a solid support, and arrays of polynucleotide probes immobilized on solid supports have been used to detect specific nucleic acid sequences in a target nucleic acid. See, e.g., PCT publication Nos. WO 89/10977 and WO 89/11548.

There is a need for improved (e.g., faster, less expensive, and more accurate) methods for characterizing the actions, targets, and toxicities of therapeutic agents in non-human primates, e.g., Cynomolgus and Rhesus monkeys during the therapeutic agent development stage.

The present invention provides a nucleotide array containing polynucleotide probes complementary to, or fragments of, any portion of a Cynomolgus monkey gene that may be used to rapidly and efficiently analyze the biological effects, including the actions, targets, and toxicities, of therapeutic agents in primates, e.g., a human, a Cynomolgus monkey or a Rhesus monkey. In one embodiment, the present invention provides a nucleotide array containing polynucleotide probes complementary to, or fragments of, any portion of a Cynomolgus monkey gene that may be used to rapidly and efficiently analyze the biological effects, including the actions, targets, and toxicities, of therapeutic agents in a non-human primate, e.g., a Cynomolgus monkey or a Rhesus monkey.

The present invention provides a nucleotide array containing polynucleotide probes complementary to, or fragments of, any portion of a Cynomolgus monkey gene, as well as polynucleotide probes complementary to, or fragments of, any portion of a Rhesus monkey gene, that may be used to rapidly and efficiently analyze the biological effects, including the actions, targets, and toxicities, of therapeutic agents in primates, e.g., a human, a Cynomolgus monkey, or a Rhesus monkey. In one embodiment, the present invention provides a nucleotide array containing polynucleotide probes complementary to, or fragments of, any portion of a Cynomolgus monkey gene, as well as polynucleotide probes complementary to, or fragments of, any portion of a Rhesus monkey gene, that may be used to rapidly and efficiently analyze the biological effects, including the actions, targets, and toxicities, of therapeutic agents in a non-human primate, e.g., a Cynomolgus monkey or a Rhesus monkey.

The present invention provides a nucleotide array containing polynucleotide probes complementary to, or fragments of, any portion of a Cynomolgus monkey gene and polynucleotide probes complementary to, or fragments of, any portion of a Rhesus monkey gene, as well as polynucleotide probes complementary to, or fragments of, any portion of a human gene, that may be used to rapidly and efficiently analyze the biological effects, including the actions, targets, and toxicities, of therapeutic agents in primates, e.g., a human, a Cynomolgus monkey, or a Rhesus monkey. In one embodiment, the present invention provides a nucleotide array containing polynucleotide probes complementary to, or fragments of, any portion of a Cynomolgus monkey gene and polynucleotide probes complementary to, or fragments of, any portion of a Rhesus monkey gene, as well as polynucleotide probes complementary to, or fragments of, any portion of a human gene, that may be used to rapidly and efficiently analyze the biological effects, including the actions, targets, and toxicities, of therapeutic agents in a non-human primate, e.g., a Cynomolgus monkey or a Rhesus monkey.

### BRIEF SUMMARY OF THE INVENTION

One aspect of the invention relates to a nucleotide array to be used in assaying gene expression upon administration of a therapeutic agent to a primate, e.g., a human, a Cynomolgus monkey, or a Rhesus monkey, especially to a non-human primate such as a Cynomolgus or a Rhesus monkey, wherein the nucleotide array comprises at least one polynucleotide probe complementary to, or a fragment of, any portion of a Cynomolgus monkey gene, such that each polynucleotide probe is immobilized to a discrete and known spot on a substrate surface. Additionally, the nucleotide array may contain at least one polynucleotide probe complementary to, or a fragment of, any portion of a member of a subset of Cynomolgus monkey genes, wherein the members of the subset are orthologs of known human genes. Furthermore, the nucleotide array may contain at least one polynucleotide probe complementary to, or a fragment of, any portion of a member of a subset of Cynomolgus monkey genes, wherein the members of the subset are homologs of known human Tox genes.

Another aspect of the invention relates to a nucleotide array to be used in assaying gene expression upon administration of a therapeutic agent to a primate, e.g., a human, a Cynomolgus monkey or a Rhesus monkey, especially to a non-human primate such as a Cynomolgus monkey or a Rhesus monkey, wherein the nucleotide array comprises at least one polynucleotide probe complementary to, or a fragment of, any portion of a Cynomolgus monkey gene and at least one polynucleotide probe complementary to, or a fragment of, any portion of a Rhesus monkey gene, such that each polynucleotide probe is immobilized to a discrete and known spot on a substrate surface. Additionally, the nucleotide array may contain at least one polynucleotide probe complementary to, or a fragment of, any portion of a member of a subset of Cynomolgus monkey genes, and optionally at least one polynucleotide probe complementary to, or a fragment of, any portion of a member of a subset of Rhesus monkey genes, wherein the members of the subsets are orthologs of known human genes. Furthermore, the nucleotide array may contain at least one polynucleotide probe complementary to, or a fragment of, any portion of a member of a subset of Cynomolgus monkey genes, and optionally a polynucleotide probe complementary to, or a fragment of, any portion of a member of a subset of Rhesus monkey genes, wherein the members of the subsets are homologs of known human Tox genes.

A third aspect of the invention relates to a nucleotide array to be used in assaying gene expression upon administration of a therapeutic agent to a primate, e.g., a human, a Cynomolgus monkey or a Rhesus monkey, especially to a non-human primate such as a Cynomolgus monkey or a Rhesus monkey, wherein the nucleotide array comprises at least one polynucleotide probe complementary to, or a fragment of, any portion of a Cynomolgus monkey gene and at least one polynucleotide probe complementary to, or a fragment of, any portion of a human gene, such that each polynucleotide probe is immobilized to a discrete and known spot on a substrate surface. Additionally, the nucleotide array may contain at least one polynucleotide probe complementary to, or a fragment of, any portion of a member of a subset of Cynomolgus monkey genes wherein the members of the subset are orthologs of known human genes. Furthermore, the nucleotide array may contain at least one polynucleotide probe complementary to, or a fragment of, any portion of a member of a subset of Cynomolgus monkey genes wherein the members of the subset are homologs of known human Tox genes. Optionally, the nucleotide array may contain at least one polynucleotide probe complementary to, or a fragment of, any portion of a human Tox genes.

A fourth aspect of the invention relates to a nucleotide array to be used in assaying gene expression upon administration of a therapeutic agent to a primate, e.g., a human, a Cynomolgus monkey, or a Rhesus monkey, especially to a non-human primate, e.g., a Cynomolgus monkey or a Rhesus monkey, wherein the nucleotide array comprises at least one polynucleotide probe complementary to, or a fragment of, any portion of a Cynomolgus monkey gene, at least one polynucleotide probe complementary to, or a fragment of any portion of a Rhesus monkey gene, and at least one polynucleotide probe complementary to, or a fragment of, any portion of a human gene, such that each polynucleotide probe is immobilized to a discrete and known spot on a substrate surface. Additionally, the nucleotide array may contain at least one polynucleotide probe complementary to, or a fragment of, any portion of a member of a subset of Cynomolgus monkey genes, and optionally at least one polynucleotide probe complementary to, or a fragment of, any portion of a member of a subset of Rhesus monkey genes, wherein the members of the subsets are orthologs of known human genes. Furthermore, the nucleotide array may contain at least one polynucleotide probe complementary to, or a fragment of, any portion of a member of a subset of Cynomolgus monkey genes, wherein the members are homologs of known human Tox genes. Optionally, the nucleotide array may contain at least one polynucleotide probe complementary to, or a fragment of, any portion of a human Tox genes and/or at least one polynucleotide probe complementary to, or a fragment of, any portion of a member of a subset of Rhesus monkey genes, wherein the members of the subset are homologs of known human Tox genes.

A fifth aspect of the invention relates to a method for identifying biomarkers upon administration of a therapeutic agent comprising administering the therapeutic agent to a primate, e.g., a human, a Cynomolgus monkey, or a Rhesus monkey, especially to a non-human primate such as a Cynomolgus monkey or a Rhesus monkey, isolating the RNA from a biological sample from the non-human primate to yield a test sample, and hybridizing the test sample with a nucleotide array containing at least one polynucleotide probe complementary to, or a fragment of, any portion of a Cynomolgus monkey gene. Optimally, the RNA from the test sample may be amplified prior to hybridization with the polynucleotide probe of the nucleotide array. According to this method, biomarkers are detected as an increased hybridization signal intensity, as compared with genes that are not affected upon administration of the therapeutic agent.

A sixth aspect of the invention relates to a method for detecting changes in gene expression upon administration of a therapeutic agent comprising administering the therapeutic agent to a primate, e.g., a human, a Cynomolgus monkey, or a Rhesus monkey, especially to a non-human primate such as a Cynomolgus monkey or a Rhesus monkey, isolating the RNA from a biological sample from the non-human primate to yield a test sample, and hybridizing the test sample with a nucleotide array containing at least one polynucleotide probe complementary to, or a fragment of, any portion of a Cynomolgus monkey gene. Optimally, the RNA from the test sample may be amplified prior to hybridization with the polynucleotide probe of the nucleotide array. According to this method, changes in gene expression are detected as alterations in the hybridization pattern for the test sample from the primate administered the therapeutic agent, as compared with the hybridization pattern for the control sample obtained from a primate that was not administered the therapeutic agent.

A seventh aspect of the invention relates to a method for identifying the targets of a therapeutic agent and/or determining the effects of a therapeutic agent on a primate, e.g., a human, a Cynomolgus monkey, or a Rhesus monkey, especially a non-human primate such as a Cynomolgus monkey or a Rhesus monkey, comprising administering the therapeutic agent to the primate of interest, isolating the RNA from a biological sample from the primate to yield a test sample, and hybridizing the test sample with a nucleotide array containing at least one polynucleotide probe complementary to, or a fragment of, any portion of a Cynomolgus monkey gene. Optimally, the RNA from the test sample may be amplified prior to hybridization with the polynucleotide probe of the nucleotide array. According to this method, the targets of a therapeutic agent and/or the effects of the therapeutic agent on the cellular metabolism are detected as alterations in the hybridization pattern for the test sample from the primate administered the therapeutic agent, as compared with the hybridization pattern for the control sample from a primate that was not administered the therapeutic agent.

An eighth aspect of the invention relates to a method of determining whether a specific gene is a target of a therapeutic agent comprising administering the therapeutic agent to a primate, e.g., a human, a Cynomolgus monkey, or a Rhesus monkey, especially to a non-human primate such as a Cynomolgus monkey or a Rhesus monkey, isolating the RNA from a biological sample of the primate to yield a test sample, and hybridizing the test sample with a nucleotide array containing at least one polynucleotide probe complementary to, or a fragment of, any portion of a Cynomolgus monkey gene. Optimally, the RNA from the test sample may be amplified prior to hybridization with the polynucleotide probe of the nucleotide array. According to this method, whether a specific gene is a target of a therapeutic agent is determined by observing the changes in the hybridization pattern for the test sample from the primate administered the therapeutic agent, as compared with the hybridization pattern for the control sample from a primate that was not administered the therapeutic agent.

A ninth aspect of the invention relates to a method of determining whether a putative target of a therapeutic agent is an actual target of a therapeutic agent comprising administering the therapeutic agent to a primate, e.g., a human, a Cynomolgus monkey, or a Rhesus monkey, especially to a non-human primate such as a Cynomolgus monkey or a Rhesus monkey, isolating the RNA from a biological sample of the primate to yield a test sample, and hybridizing the test sample with a nucleotide array containing at least one polynucleotide probe complementary to, or a fragment of, any portion of a Cynomolgus monkey gene. Optimally, the RNA from the test sample may be amplified prior to hybridization with the polynucleotide probe of the nucleotide array. According to this method, whether a putative drug target is an actual drug target is determined by observing the changes in the hybridization pattern for the test sample from the primate administered the therapeutic agent, as compared with the hybridization pattern for the control sample from a primate that was not administered the therapeutic agent.

A tenth aspect of the invention relates to a method of determining a more target-specific therapeutic agent from an initial therapeutic agent comprising: (a) determining the targets of the initial therapeutic agent by administering the therapeutic agent to a primate, e.g., a human, a Cynomolgus monkey, or a Rhesus monkey, especially to a non-human primate such as a Cynomolgus monkey or a Rhesus monkey, isolating the RNA from a biological sample from the primate to yield a test sample, and hybridizing the test sample with a nucleotide array containing at least one polynucleotide probe complementary to, or a fragment of, any portion of a Cynomolgus monkey gene; (b) modifying the structure of the initial therapeutic agent; and (c) determining the targets of the modified initial therapeutic agent by administering the therapeutic agent to a primate, e.g., a human, a Cynomolgus monkey, or a Rhesus monkey, especially to a non-human primate such as a Cynomolgus monkey or a Rhesus monkey, isolating the RNA from a biological sample from the primate to yield a test sample, and hybridizing the test sample with a nucleotide array containing at least one polynucleotide probe complementary to, or a fragment of, any portion of a Cynomolgus monkey gene. Optimally, the RNA from the test sample may be amplified prior to hybridization with the polynucleotide probe of the nucleotide array.

An eleventh aspect of the invention relates to a method of identifying single nucleotide polymorphisms comprising isolating the RNA from a biological sample of a primate, e.g., a human, a Cynomolgus monkey, or a Rhesus monkey, especially to a non-human primate such as a Cynomolgus monkey or a Rhesus monkey, to yield a test sample, and hybridizing the test sample with a nucleotide array containing at least one polynucleotide probe complementary to, or a fragment of, any portion of a Cynomolgus monkey gene. Optimally, the RNA from the test sample may be amplified prior to hybridization with the polynucleotide probe of the nucleotide array. According to this method, a single nucleotide polymorphism would be identified by observing the decreases in intensity of the hybridization pattern for a primate that has a single nucleotide polymorphism as compared with the intensity of the hybridization pattern for a primate without the single nucleotide polymorphism.

A twelfth aspect of the invention relates to a method of normalizing data comprising (a) determining the signal intensity of the hybridized complex between the test or control sample and a polynucleotide probe that is complementary to, or a fragment of, a Cynomolgus monkey gene that is known not to be up- or down-regulated upon administration of a specific therapeutic agent; (b) averaging the signal intensity of the specific hybridized complex on different nucleotide arrays; (c) determining the ratio between the average signal intensity of the specific hybridized complex on all of the nucleotide arrays and the signal intensity of the specific hybridized complex on the nucleotide array of interest; and (d) adjusting the signal intensities of the hybridized complexes between the other hybridized complexes on the nucleotide array based upon the calculated ratio.

A thirteenth aspect of the invention relates to an in vitro system utilizing a nucleotide array containing a polynucleotide probe that is complementary to, or a fragment of, any portion of a Cynomolgus monkey gene. The gene expression upon exposure of a therapeutic agent to an isolated cell line may be assayed by exposing the therapeutic agent to a cell line isolated from a primate, e.g., a cell line isolated from a human, a cell line isolated from a Cynomolgus monkey, or a cell line isolated from a Rhesus monkey, especially a cell line isolated from a non-human primate such as a Cynomolgus monkey or a Rhesus monkey, isolating the RNA from the cell line to yield a test sample, and hybridizing the test sample with the nucleotide array of interest. In one embodiment, the changes in gene expression are detected by comparing the hybridization pattern of the nucleotide array exposed to the test sample of a cell line from a primate exposed to a therapeutic agent with the hybridization pattern of a nucleotide array exposed to the control sample of a cell line from a primate that was not exposed to a therapeutic agent.

### DETAILED DESCRIPTION OF THE INVENTION

Terminology

As used herein, the term “gene” refers to a deoxyribonucleic acid (“DNA”) sequence comprising several operably linked DNA fragments such as a promoter region, a 5′ untranslated region (“5′UTR”), a coding region (which may or may not code for a protein), and an untranslated 3′ region (“3′UTR”) comprising a polyadenylation site. Typically, the 5′UTR, the coding region and the 3′UTR are transcribed into a ribonucleoprotein (“RNA”) of which, in the case of a protein encoding gene, the coding region is translated into a protein. A gene may include additional DNA fragments such as, for example, introns.

As used herein, the term “nucleic acid” or “nucleic acid molecule” refers to a polymeric form of nucleotides of any length, either ribonucleotides or deoxyribonucleotides, in either single- or double-stranded from, and that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components.

As used herein, the terms “polynucleotide probe” or “polynucleotide probes” refers to an oligodeoxyribonucleotide or oligoribonucleotide, or any modified form of these polymers that are capable of hybridizing with a target nucleic acid sequence by complementary base-pairing. Complementary base pairing means sequence-specific base pairing that includes e.g., Watson-Crick base pairing as well as other forms of base pairing such as Hoogsteen base pairing. Modified forms include 2′-O-methyl oligoribonucleotides and so-calls PNAs, in which oligodeoxyribonucleotides are linked via peptide bonds rather than phosphodiester bonds.

As used herein, the term “nucleotide array” refers to a multiplicity of different polynucleotide probes attached (preferably through a single terminal covalent bond) to one or more solid supports where, when there is a multiplicity of supports, each support bears a multiplicity of polynucleotide probes. The term “nucleotide array” can refer to the entire collection of polynucleotide probes on the support(s) or to a subset thereof. The spatial distribution of the polynucleotide probes may differ between two or more nucleotide arrays, but in a preferred embodiment, the spatial distribution is substantially the same. It is recognized that even where two nucleotide arrays are designed and synthesized to be identical there are variations in the abundance, composition, and distribution of the polynucleotide probes. These variations are preferably insubstantial and/or compensated for by the use of controls.

As used herein, the term “complementary to” refers to sequence that will form specific base pairing that includes e.g., Watson-Crick base pairing as well as other forms of base pairing such as Hoogsteen base pairing, with a nucleic acid of interest.

As used herein, the term “fragment of” refers to an oligodeoxyribonucleotide, oligoribonucleotide, a DNA, RNA, or other nucleic acid molecule or any modified form of these polymers, wherein the polymer is shorter than the full-length gene of interest, wherein these polymers are capable of hybridizing with a target nucleic acid sequence by complementary base pairing. Complementary base pairing means sequence-specific base pairing that includes e.g., Watson-Crick base pairing as well as other forms of base pairing such as Hoogsteen base pairing. Modified forms include 2′-O-methyl oligoribonucleotides and so-called PNAs, in which oligodeoxyribonucleotides are linked via peptide bonds rather than phosphodiester bonds. Examples of suitable lengths of the fragment may include, but not limited to, about 100 nucleotides in length, about 10 to about 50 nucleotides in length, about 15 to about 45 nucleotides in length, about 20 to about 40 nucleotides in length, about 20 to about 35 nucleotides in length, about 20 to about 30 nucleotides in length, about 22 to about 27 nucleotides in length, or about 25 nucleotides in length.

As used herein, the term “primate” refers to a mammal that falls within the human, ape or monkey family and includes, but is not limited to, a human, a baboon, a chimpanzee, a capuchin, a pigtail macaque, a sooty mangabey, a squirrel monkey, a gibbon, a Rhesus monkey, a Cynomolgus monkey, a gorilla, an orangutan, and any other ape or monkey species.

As used herein, the term “non-human primate” refers to a mammal that falls within the ape or monkey family and includes, but is not limited to, a baboon, a chimpanzee, a capuchin, a pigtail macaque, a sooty mangabey, a squirrel monkey, a gibbon, a Rhesus monkey, a Cynomolgus monkey, a gorilla, an orangutan, and any other ape or monkey species.

As used herein, the terms “therapeutic agent” or “therapeutic agents” refers to any compounds which have an effect on a cell or tissue. The therapeutic agent need not have any proven therapeutic benefit. Therapeutic agents include: typical small molecules of research or therapeutic interest; naturally-occurring factors, such as proteins, including antibodies, receptors, ligands, endocrine, paracrine, or autocrine factors or factors interacting with cell receptors of all types; intracellular factors, such as elements of intracellular signaling pathways; and factors isolated from other natural sources, such as carbohydrates, e.g., sugars, and lipids.

As used herein, the term “biological sample” refers to any blood or tissue sample obtained from a primate, e.g., a human, a Cynomolgus monkey, or a Rhesus monkey, including but not limited to, samples obtained from the liver, lung, lymph node, kidney, bone marrow, thymus, heart, kidney, spleen, brain, or serum.

As used herein, the term “test sample” refers to total cellular RNA directly isolated from, including mRNA, or a nucleic acid complementary to, or a fragment of, the RNA isolated from, a biological sample obtained from a primate, e.g., a human, a Cynomolgus monkey or a Rhesus monkey, especially from a non-human primate such as a Cynomolgus monkey or a Rhesus monkey, administered a therapeutic agent. Alternatively, the test sample may be either total cellular RNA directly isolated from, including mRNA, or a nucleic acid complementary to, or a fragment of, the RNA isolated from, a biological sample obtained from a primate, e.g., a human, a Cynomolgus monkey, or a Rhesus monkey, especially from a non-human primate such as a Cynomolgus monkey or a Rhesus monkey, that has a single nucleotide polymorphism. The difference in usage will be apparent from the context.

The test sample includes, but is not limited to isolated RNA, including mRNA, a cDNA reverse transcribed from the isolated RNA, an RNA transcribed from the cDNA, a DNA amplified from the cDNA, and an RNA transcribed from the amplified DNA.

As used herein, the term “control sample” refers to the total cellular RNA directly isolated from, including mRNA, or a nucleic acid complementary to, or a fragment of, the RNA isolated from, a biological sample obtained from a primate, e.g., a human, a Cynomolgus monkey, or a Rhesus monkey, especially from a non-human primate such as a Cynomolgus monkey or Rhesus monkey, that was not administered a therapeutic agent. Alternatively, the control sample may be either the total cellular RNA directly isolated from, including mRNA, or a nucleic acid complementary to, or a fragment of, the RNA isolated from, a biological sample obtained from a primate, e.g., a human, a Cynomolgus monkey, or a Rhesus monkey, especially from a non-human primate such as a Cynomolgus monkey or a Rhesus monkey that lacks the single nucleotide polymorphism. The difference in usage will be apparent from the context.

The control sample includes, but is not limited to isolated RNA, including mRNA, a cDNA reverse transcribed from the isolated RNA, an RNA transcribed from the cDNA, a DNA amplified from the cDNA, and an RNA transcribed from the amplified DNA.

As used herein, the term “homolog” refers to a gene that is related to a second gene by descent from a common ancestral sequence. The term may apply to the relationship between genes of two different species that have the same function. Additionally, the term may apply to genes, within the same species, that are related through genetic duplication, but have different functions.

As used herein, the term “homologous” refers to the relationship between two species wherein one gene is related to a second gene by descent from a common ancestral sequence. The term may apply to the relationship between genes of two different species that have the same function. Additionally, the term may apply to the relationship between genes that are related through genetic duplication but have different functions.

As used herein, the term “ortholog” refers to that subset of homologous genes which encompasses a gene in two different species that evolved from a common ancestor that has the same function in both species.

As used herein, the term “expressed sequence tags” (“ESTs”) encompasses pieces of DNA that are a copy of the gene that is expressed. ESTs may be a copy of either the 5′ or 3′ end of the gene. ESTs may be prepared by reverse transcribing mRNA that was isolated from a tissue and then inserting the reverse transcription product into a vector. Generally, ESTs are about 200 to about 750 nucleotides long.

The term “target nucleic acid” refers to a nucleic acid to which a polynucleotide probe is designed to specifically hybridize. It is either the presence or absence of the target nucleic acid that is to be detected, or the amount of the target nucleic acid that is to be quantified utilizing a nucleotide array of the present invention. The target nucleic acid has a sequence that is complementary to, or is a fragment of, the nucleic acid sequence of the corresponding polynucleotide probe directed to the target nucleic acid. The term target nucleic acid may refer to the specific subsequence of a larger nucleic acid to which the polynucleotide probe is directed or to the overall sequence (e.g., gene or RNA) whose expression level it is desired to detect. The difference in usage will be apparent from context.

As used herein, the term “mismatch control” or “mismatch controls” refers to a polynucleotide probe that has a sequence deliberately selected not to be perfectly complementary to, or is a fragment of, a particular target nucleic acid. The mismatch control typically has a corresponding test polynucleotide probe that is perfectly complementary to, or is a fragment of, the sequence of the same particular target nucleic acid except for the presence of one or more mismatched bases. A mismatched base is a base selected so that it is not complementary to, or is not a fragment of, the corresponding base in the sequence of the target nucleic acid to which the polynucleotide probe would otherwise specifically hybridize. One or more mismatches are selected such that under appropriate hybridization conditions (e.g., stringent conditions), the test or control polynucleotide probe would be expected to hybridize with its target nucleic acid, but the mismatch polynucleotide probe would not hybridize (or would hybridize to a significantly lesser extent). In one embodiment, the mismatch polynucleotide probe would contain a central mismatch. Thus, for example, where a polynucleotide probe contains 20 nucleotides, a corresponding mismatch polynucleotide probe will have the identical sequence except for a single mismatch base (e.g., substituting a G, a C, or a T for an A) at any of positions 6 through 14 (the central mismatch).

As used herein, the term “normalization control” or “normalization controls” refers to a polynucleotide probe that has a sequence deliberately selected to be to perfectly complementary to, or is a fragment of, a labeled target nucleic acid added to the test or control sample. The signals obtained from the normalization controls after hybridization provide a control for variations in hybridization conditions, label intensity, “reading” efficiency, and other factors that may cause the signal of a perfect hybridization to vary between nucleotide arrays. In one embodiment, signals (e.g., fluorescence intensity) read from all other polynucleotide probes in the nucleotide array are divided by the signal (e.g., fluorescence intensity) from the control polynucleotide probes, thereby normalizing the measurements.

As used herein, the term “expression level control” or “expression level controls” refers to a polynucleotide probe that has a sequence deliberately selected to be perfectly complementary to, or is a fragment of, a constitutively expressed gene in the test sample. Expression level controls are designed to control for the overall health and metabolic activity of a cell. Examination of the covariance of an expression level control with the expression level of the target nucleic acid indicates whether measured changes or variations in expression level of a gene is due to changes in transcription rate of that gene or to general variations in health of the cell. Thus, for example, when a cell is in poor health, or lacking a critical metabolite, the expression levels of both an active gene and a constitutively expressed gene are expressed to decrease. The converse is also true. Thus, where the expression levels of both an expression level control and the gene appear to both decrease or to both increase, the change may be attributed to changes in the metabolic activity of the cell as a whole, not to differential expression of the gene in question. Conversely, where the expression levels of the gene and the expression level control do not covary, the variation in the expression level of the gene is attributed to differences in regulation of that gene and not to variations in the metabolic activity of the cell.

As used herein, the term “sample preparation/amplification control” or “sample preparation/amplification controls” refers to a polynucleotide probe that is complementary to, or is a fragment of, subsequences of control genes selected because they do not normally occur in the nucleic acids of the particular biological sample being assayed. Suitable sample preparation/amplification controls include, for example, polynucleotide probes to bacterial genes (e.g., Bio B).

The term “solid support,” “support,” or “substrate” refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments the solid support(s) will take the form of beads, resins, gels, microspheres, or other geometric configurations.

Examples of suitable substrates include, but are not limited to, silicon or glass. The silicon or glass substrate may have the thickness of a microscope slide or glass cover slip. Substrates that are transparent to light are useful when the assay involves optical detection. Other useful substrates include Langmuir Blodgett film, germanium, (poly)tetrafluorethylene, polystyrene, (poly)vinylidenedifluoride, polycarbonate, gallium arsenide, gallium phosphide, silicon oxide, silicon dioxide, silicon nitride, and combinations thereof.

As used herein, the term “background,” “background signal,” or “background signals” refers to the hybridization signal resulting from non-specific binding, or other interactions, between a labeled target nucleic acid and components of a nucleotide array (e.g., the polynucleotide probes, mismatch controls, or the nucleotide array support). Background signals may also be produced by intrinsic fluorescence of the nucleotide array components themselves. A single background signal can be calculated for the entire nucleotide array, or a different background signal may be calculated for each target nucleic acid. In one embodiment, background is calculated as the average hybridization signal intensity for the lowest 5% to 10% of the polynucleotide probes in the nucleotide array. Of course, a person of ordinary skill in the art will appreciate that where the polynucleotide probes hybridize well, and thus appear to be specifically binding to a target nucleic acid, those polynucleotide probes should not be used in a background signal calculation. Alternatively, background may be calculated as the average hybridization signal intensity produced by hybridization to polynucleotide probes that are not complementary to, or are not fragments of, any sequence found in the test or control sample (e.g. polynucleotide probes directed to nucleic acids of the opposite sense or to genes not found in the test or control sample, such as bacterial genes). Background may also be calculated as the average signal intensity produced by regions of the nucleotide array that lack any polynucleotide probes at all.

As used herein, the term “hybridizing specifically to” refers to the binding, duplexing, or hybridizing of a nucleic acid only to a particular polynucleotide probe under stringent conditions when that nucleic acid is present in either the test or control sample.

As used herein, the term “stringent conditions” refers to conditions under which a polynucleotide probe will hybridize to its target nucleic acid, but to no other nucleic acids. Stringent conditions are sequence-dependent and will be different in different circumstances. Longer polynucleotide probes hybridize specifically at higher temperatures. Generally, stringent conditions are selected to be about 5° C. lower than the thermal melting point (“Tₘ”) for the specific polynucleotide probes at a defined ionic strength and pH.

As used herein, “Tₘ” is the temperature (under defined ionic strength, pH, and nucleic acid concentration) at which 50% of the polynucleotide probes complementary to, or fragments of, the sequences of the target nucleic acid hybridize to the target nucleic acid at equilibrium. Because the target nucleic acids are generally present in excess, at Tₘ, 50% of the polynucleotide probes are occupied at equilibrium. Typically, stringent conditions will be those in which the salt concentration is at least about 0.01 to 1.0 M Na ion concentration (or other salts), pH 7.0 to 8.3, and a temperature of at least about 30° C. for short polynucleotide probes (e.g., 10 to 50 nucleotides). Stringent conditions may also be achieved with the addition of destabilizing agents, such as formamide or tetralkyl ammonium salts.

As used herein, the term “perfectly matched polynucleotide probe” or “perfectly matched polynucleotide probes” refers to a nucleic acid that has a sequence perfectly complementary to, or is a fragment of, the sequence of a particular target nucleic acid. Such a polynucleotide probe is typically perfectly complementary to, or is a fragment of, a portion (subsequence) of the target nucleic acid.

As used herein, the term “polymorphic marker” or “polymorphic site” is the locus at which divergence occurs. Preferred polymorphic markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as a reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

A single nucleotide polymorphism (“SNP”) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. SNP's may also arise from a deletion of a nucleotide or an insertion of a nucleotide relative to a reference allele.

As used herein, the term “distinct” means that each row of the nucleotide array is separated by some physical distance.

As used herein, the term “immobilized” means that the polynucleotide probe is attached to a solid support, preferably covalently.

As used herein, the term “biomarker” or “marker” encompasses a broad range of intra- and extra-cellular events as well as whole-organism physiological changes. Biomarkers may be represent essentially any aspect of cell function, for example, but not limited to, levels or rate of production of signaling molecules, transcription factors, metabolites, gene transcripts as well as post-translational modifications of proteins. Biomarkers may include whole genome analysis of transcript levels or whole proteome analysis of protein levels and/or modifications.

A biomarker may also refer to a gene or gene product which is up- or down-regulated in a therapeutic agent-treated, diseased cell of a primate having the disease compared to an untreated diseased cell. That is, the gene or gene product is sufficiently specific to the treated cell that it may be used, optionally with other genes or gene products, to identify, predict, or detect efficacy of a small molecule. Thus, a biomarker is a gene or gene product that is characteristic of efficacy of a compound in a diseased cell or the response of that diseased cell to treatment by the compound.

Biomarkers may indicate whether a particular therapeutic agent may be toxic, may predict whether an individual primate will respond to a therapeutic agent, or whether a therapeutic agent will be efficacious.

The present invention provides a nucleotide array containing polynucleotide probes complementary to, or fragments of, any portion of a Cynomolgus monkey gene and a method of using such a nucleotide array to characterize the biological effects, including the actions, targets, and toxicities, of therapeutic agents in a primate, e.g., a human, a Cynomolgus monkey, or a Rhesus monkey, including identifying the targets of the therapeutic agents, improving lead compounds, and investigating the toxicities of therapeutic agents. In one embodiment, the present invention provides a nucleotide array containing polynucleotide probes complementary to, or fragments of, any portion of a Cynomolgus monkey gene and a method of using such a nucleotide array to characterize the biological effects, including the actions, targets, and toxicities, of therapeutic agents in a non-human primate, e.g., a Cynomolgus monkey or a Rhesus monkey, including identifying the targets of the therapeutic agents, improving lead compounds, and investigating the toxicities of therapeutic agents.

The biological effect of a therapeutic agent may be a consequence of, inter alia, therapeutic agent-mediated changes in the rate of transcription or degradation of one or more species of RNA, the rate or extent of translation or post-translational processing of a polypeptide, the rate or extent of protein degradation, and the inhibition or stimulation of protein action or activity.

The biological effects of a therapeutic agent are detected in the instant invention by measuring and/or observing the biological state of a cell or tissue exposed to the therapeutic agent or a metabolite of the therapeutic agent. The biological state of a cell is the state of a collection of cellular constituents that are sufficient to characterize the effects of a drug.

One aspect of the biological state of a cell or tissue that may be measured or observed in the present invention is the transcriptional state of a cell or tissue. The transcriptional state of a cell or tissue is the identities and abundances of the constituent RNA species, especially mRNAs, in the cell under a given set of conditions.

Administration of a therapeutic agent may affect the transcriptional state of a cell or tissue in a variety of ways. The administration of a therapeutic agent may result in a change, through direct or indirect effects, in the transcriptional state of a cell. In certain instances, the effect of the therapeutic agent may be either up- or down-regulation of the transcriptional state.

One reason that exposure to a therapeutic agent results in a change to the transcriptional state of a cell or tissue is that the feedback systems which react in a compensatory manner to administration of a therapeutic agent do so primarily by altering patterns of gene expression or transcription. Because the changes in the transcriptional state of a cell or tissue may be profound, this invention provides a method by which controlled measurements and/or observations of the biological state of a cell or tissue may be made to determine the effects or the direct targets of a therapeutic agent in a primate, e.g., a human, a Cynomolgus monkey, or a Rhesus monkey. In one embodiment, the measurements and/or observations of the effects of a therapeutic agent may be utilized to analyze the actions, targets, and toxicities of therapeutic agents in a non-human primate, e.g., a Cynomolgus monkey or a Rhesus monkey, and to thereby identify which therapeutic agents warrant further development, e.g., clinical testing in humans.

Generally, the methods of observing the biological effects of a therapeutic agent utilizing a nucleotide array involves preparing the nucleotide array, providing a pool of target nucleic acids comprising RNA transcripts, or nucleic acids complementary to, or fragments of, the RNA transcripts, from a test and/or control sample, hybridizing the test and/or control sample to the nucleotide array (including control polynucleotide probes) and detecting the hybridized nucleic acid complexes.

Nucleotide Array Design

A person of ordinary skill in the art will appreciate that a number of possible nucleotide array designs are suitable for the practice of this invention. The nucleotide array will typically include one or more polynucleotide probes that specifically hybridize to the target nucleic acid. In addition, the nucleotide array may optionally include one or more normalization controls, expression level controls, mismatch controls, and/or sample preparation/amplification controls.

1) Polynucleotide Probes

Certain polynucleotide probes incorporated onto a nucleotide array to be utilized in the methods of the present invention are complementary to, or fragments of, portions of ESTs of the Cynomolgus monkey. Additionally, a nucleotide array utilized in the methods of the present invention may optionally include polynucleotide probes complementary to, or fragments of, portions of ESTs of the Rhesus monkey. Furthermore, a nucleotide array that may be utilized in the methods of the present invention may optionally include polynucleotide probes complementary to, or fragments of, portions of human genes. Optionally, the nucleotide array may include fragments of Cynomolgus monkey, Rhesus monkey, and/or human genomic DNA.

Cynomolgus monkey ESTs were identified by isolating mRNA in the liver, lung, lymph node, kidney, bone marrow, thymus, heart, kidney, spleen, and brain. The isolated mRNAs were amplified using polymerase chain reaction (“PCR”) and then sequenced. The sequenced ESTs from Cynomolgus monkey were used to generate the polynucleotide probes to be immobilized to the nucleotide array. The sequences of the ESTs from Cynomolgus monkey are identified herein as SEQ ID NOS. 1-8881 and 9187-18598. Certain of SEQ ID NOS. 9187-18598, as indicated in the Sequence Listing, are orthologs of known human genes.

SEQ ID NOS 17249-18598, as indicated in the Sequence Listing, are Cynomolgus genes that are homologous to known human Tox genes. The human Tox genes have previously been identified as being activated in response to a toxic therapeutic agent. Therefore, a nucleotide array containing polynucleotide probes complementary to, or fragments of, any portion of SEQ ID NOS. 17249-18598 will be useful in exploring the toxicity of a therapeutic agent when administered to a non-human primate, e.g., a Cynomolgus or a Rhesus monkey.

Additionally, a nucleotide array to be utilized in the methods of the present invention may optionally include polynucleotide probes complementary to, or fragments of, any portion of an EST of the Rhesus monkey. Also, a nucleotide array to be utilized in the methods of the present invention may optionally include polynucleotide probes complementary to, or fragments of, any portion of a genomic sequence of the Rhesus monkey. The ESTs and genomic sequences from the Rhesus monkey used to generate the polynucleotide probes immobilized to the nucleotide array of the present invention are identified as SEQ ID NOS. 18599-35840 and SEQ ID NOS. 36075-43225. Certain of SEQ ID NOS. 18599-35840, as indicated in the Sequence Listing, are orthologs of known human genes.

SEQ ID NOS 18599-20526, as indicated in the Sequence Listing, are Rhesus genes that are homologous to known human Tox genes. The human Tox genes have been previously identified as being activated in response to a toxic therapeutic agent. Therefore, a nucleotide array containing polynucleotide probes complementary to, or fragments of, any portion of SEQ ID NOS. 18599-20526 will be useful in exploring the toxicity of a therapeutic agent when administered to a non-human primate, e.g., a Cynomolgus or a Rhesus monkey.

Furthermore, the nucleotide array to be utilized in the methods of the present invention may optionally include polynucleotide probes complementary to, or fragments of, any portion of a human gene. The human sequences used to generate the polynucleotide probes immobilized to the nucleotide array of the present invention are identified as SEQ ID NOS. 43450-48714.

The polynucleotide probes complementary to, or fragments of, any portion of SEQ ID NOS. 43450-48714 were selected to be complementary to, or fragments of, human genes in which no homologous genomic sequence has been identified in the Cynomolgus or Rhesus monkey.

Therefore, the polynucleotide probes identified as SEQ ID NOS. 8882-9186 and 35841-36074 and the polynucleotide probes complementary to, or fragments of, any portion of SEQ ID NOS. 1-8881, SEQ ID NOS. 9187-18598, SEQ ID NOS. 18599-35840 or SEQ ID NOS. 36075-43225 to be immobilized to the nucleotide array to be utilized in the methods of the present invention were selected to exhibit greater complementarity with both the Cynomolgus and Rhesus monkey genome, than seen with a nucleotide array using polynucleotide probes generated solely from human genomic sequences. The greater sequence complementarity will yield a more effective nucleotide array to examine the actions, targets, and toxicities of therapeutic agents in Cynomolgus or Rhesus monkey than a nucleotide array containing polynucleotide probes generated solely from human genomic sequences.

The polynucleotide probes to be included on the nucleotide array of the prevent invention, SEQ ID NOS. 8882-9186, SEQ ID NOS. 35841-36074, and/or SEQ ID NOS. 43226-43449, or polynucleotide probes that are complementary to, or fragments of, SEQ ID NOS. 1-8881, SEQ ID NOS. 9187-18598, SEQ ID NOS. 18599-35840, SEQ ID NOS. 36075-43225, and/or SEQ ID NOS. 43450-48714, may be oligodeoxyribonucleotides or oligoribonucleotides, or any modified forms of these polymers that are capable of hybridizing with a target nucleic sequence by complementary base-pairing. Complementary base pairing means sequence-specific base pairing which includes e.g., Watson-Crick base pairing as well as other forms of base pairing such as Hoogsteen base pairing. Modified forms include 2′-O-methyl oligoribonucleotides and so-called PNAs, in which oligodeoxyribonucleotides are linked via peptide bonds rather than phosphodiester bonds.

The polynucleotide probes complementary to, or fragments of, any portion of SEQ ID NOS. 1-8881, SEQ ID NOS. 9187-18598, SEQ ID NOS. 18599-35840, SEQ ID NOS. 36075-43225, or SEQ ID NOS. 43450-48714 may be a range of nucleotide lengths. The polynucleotide probes may be as long as the number of nucleotides of the EST or genomic fragment. The polynucleotide probes may be as long as about 100 nucleotides. Optionally, the polynucleotide probes complementary to, or fragments of, any portion of SEQ ID NOS. 1-8881, SEQ ID NOS. 9187-18598, SEQ ID NOS. 18599-35840, SEQ ID NOS. 36075-43225, or SEQ ID NOS. 43450-48714 may be from about 10 to about 50 nucleotides in length. In one embodiment, the polynucleotide probes complementary to, or fragments of, any portion of SEQ ID NOS. 1-8881, SEQ ID NOS. 9187-18598, SEQ ID NOS. 18599-35840, SEQ ID NOS. 36075-43225, and SEQ ID NOS. 43450-48714 may be from about 15 to about 45 nucleotides in length. In another embodiment, the polynucleotide probes complementary to, or fragments of, any portion of SEQ ID NOS. 1-8881, SEQ ID NOS. 9187-18598, SEQ ID NOS. 18599-35840, SEQ ID NOS. 36075-43225, or SEQ ID NOS. 43450-48714 may be from about 20 to about 40 nucleotides in length. In yet another embodiment, the polynucleotide probes complementary to, or fragments of, any portion of SEQ ID NOS. 1-8881, SEQ ID NOS. 9187-18598, SEQ ID NOS. 18599-35840, SEQ ID NOS. 36075-43225, or 43450-48714 may be from about 20 to about 35 nucleotides in length. In still yet another embodiment, the polynucleotide probes complementary to, or fragments of, any portion of SEQ ID NOS. 1-8881, SEQ ID NOS. 9187-18598, SEQ ID NOS. 18599-35840, SEQ ID NOS. 36075-43225, or 43450-48714 may be from about 20 to about 30 nucleotides in length. In another embodiment, the polynucleotide probes complementary to, or fragments of, any portion of SEQ ID NOS. 1-8881, SEQ ID NOS. 9187-18598, SEQ ID NOS. 18599-35840, SEQ ID NOS. 36075-43225, and SEQ ID NOS. 43450-48714 may be from about 22 to about 27 nucleotides in length. In still yet another embodiment, the polynucleotide probes complementary to, or fragments of, any portion of SEQ ID NOS. 1-8881, SEQ ID NOS. 9187-185988, SEQ ID NOS. 18599-35840, SEQ ID NOS. 36075-43225, or SEQ ID NOS. 43450-48714 may be about 25 nucleotides in length.

An embodiment of the invention has at least one polynucleotide probe complementary to, or a fragment of, any portion of a sequence identified in the Cynomolgus monkey immobilized to a discrete and known spot on a solid support. In one embodiment of the invention, the nucleotide array has at least one polynucleotide probe that is complementary to, or a fragment of, any portion of an ortholog, of a human gene, immobilized to a discrete and known spot on a solid support. In another embodiment, the nucleotide array may include at least one polynucleotide probe complementary to, or a fragment of, any portion of an ortholog to a human Tox gene immobilized to a discrete and known spot on a solid support. In yet another embodiment, the nucleotide array may include at least one polynucleotide probe complementary to, or a fragment of, any portion of SEQ ID NOS. 1-8881 or SEQ ID NOS. 9187-18598, Cynomolgus genes, immobilized to a discrete and known spot on a solid support. In still yet another embodiment, the nucleotide array may include a polynucleotide probe complementary to, or a fragment of, any portion of only SEQ ID NOS. 9187-18598 immobilized to a discrete and known spot on a solid support. In another embodiment, the nucleotide array may include at least one polynucleotide probe complementary to, or a fragment of, any portion of SEQ ID NOS. 17249-18598 immobilized to a discrete and known spot on a solid support. In yet another embodiment, the nucleotide array may include any one of SEQ ID NOS. 8882-9186 as the polynucleotide probes immobilized to a discrete and known spot on a solid support.

Another embodiment of the invention has at least one polynucleotide probe complementary to, or a fragment of, any portion of a sequence identified in the Cynomolgus monkey, as well as at least one polynucleotide probe complementary to, or a fragment of, any portion of a human gene, immobilized to a discrete and known spot on a solid support. In one embodiment of the invention, the polynucleotide probe from the human gene is complementary to, or a fragment of, any portion of any of SEQ ID NOS. 43450-48714. In another embodiment of the invention, the nucleotide array may include any one of SEQ ID NOS. 43226-48714 as the polynucleotide probes immobilized to a discrete and known spot on a solid support. In still yet another embodiment of the invention, the nucleotide array may include at least one polynucleotide probe complementary to, or a fragment of, any portion of a human gene in combination with any of the polynucleotide probes, described above, directed to a Cynomolgus gene.

Yet another embodiment of the invention has at least one polynucleotide probe complementary to, or a fragment of, any portion of a sequence identified in the Cynomolgus monkey, as well as at least one polynucleotide probe complementary to, or fragment of, any portion of a gene from a Rhesus monkey, immobilized to a discrete and known spot on a solid support. In one embodiment of the invention, the polynucleotide probe from the Rhesus monkey gene is complementary to, or a fragment of, any portion of any of SEQ ID NOS. 18599-35840 and SEQ ID NOS. 36075-43225. In another embodiment of the invention, the polynucleotide probe from the Rhesus monkey gene is complementary to, or a fragment of, any portion of any of SEQ ID NOS. 18599-20526. In yet another embodiment of the invention, the nucleotide array may include any one of SEQ ID NOS. 35841-36074 as the polynucleotide probes immobilized to a discrete and known spot on a solid support. In still yet another embodiment of the invention, the nucleotide array may include at least one polynucleotide probe complementary to, or a fragment of, any portion of a Rhesus monkey gene in combination with any of the polynucleotide probes, described above, directed to a Cynomolgus gene.

Still yet another embodiment of the invention has at least one polynucleotide probe complementary to, or a fragment of, any portion of a sequence identified in the Cynomolgus monkey, as well as at least one polynucleotide probe complementary to, or a fragment of, any portion of a gene from a Rhesus monkey and at least one polynucleotide probe complementary to, or a fragment of, any portion of a human gene, immobilized to a discrete and known spot on a solid support. In one embodiment of the invention, the nucleotide array has at least one polynucleotide probe that is complementary to, or a fragment of, any portion of an ortholog identified in the Cynomolgus monkey, of a human gene, as well as at least one polynucleotide probe complementary to, or a fragment of, any portion of a Rhesus monkey gene and at least one polynucleotide probe complementary to, or a fragment of, any portion of any human gene, immobilized to a discrete and known spot on a solid support. In another embodiment of the invention, the nucleotide array has at least one polynucleotide probe that is complementary to, or a fragment of, any portion of an ortholog identified in the Cynomolgus monkey, of a human gene, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is complementary to, or a fragment of, any portion of any of SEQ ID NOS. 43450-48714, immobilized to a discrete and known spot on a solid support. In yet another embodiment of the invention, the nucleotide array has at least one polynucleotide probe that is complementary to, or a fragment of, any portion of an ortholog identified in the Cynomolgus monkey, of a human gene, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is any of SEQ ID NOS. 43226-48714, immobilized to a discrete and known spot on a solid support. In still yet another embodiment of the invention, the nucleotide array has at least one polynucleotide probe that is complementary to, or a fragment of, any portion of an ortholog identified in the Cynomolgus monkey, of a human gene, where at least one polynucleotide probe from a Rhesus monkey gene is complementary to, or a fragment of, any portion of any of SEQ ID NOS. 18599-20526 and at least one polynucleotide probe from the human gene is complementary to, or a fragment of, any portion of any of SEQ ID NOS. 43450-48714, immobilized to a discrete and known spot on a solid support. In one embodiment of the invention, the nucleotide array has at least one polynucleotide probe that is complementary to, or a fragment of, any portion of an ortholog identified in the Cynomolgus monkey, of a human gene, where at least one polynucleotide probe from a Rhesus monkey gene is complementary to, or a fragment of, any portion of any of SEQ ID NOS. 18599-20526 and at least one polynucleotide probe from the human gene is any of SEQ ID NOS. 43226-48714, immobilized to a discrete and known spot on a solid support. In another embodiment of the invention, the nucleotide array has at least one polynucleotide probe that is complementary to, or a fragment of, any portion of an ortholog identified in the Cynomolgus monkey, of a human gene, where at least one polynucleotide probe from a Rhesus monkey gene is any of SEQ ID NOS. 35841-36074 and at least one polynucleotide probe from the human gene is complementary to, or a fragment of, any portion of any of SEQ ID NOS. 43450-48714, immobilized to a discrete and known spot on a solid support. In yet another embodiment of the invention, the nucleotide array has at least one polynucleotide probe that is complementary to, or a fragment of, any portion of an ortholog identified in the Cynomolgus monkey, of a human gene, where at least one polynucleotide probe from a Rhesus monkey gene is any of SEQ ID NOS. 35841-36074 and at least one polynucleotide probe from the human gene is any of SEQ ID NOS. 43226-48714, immobilized to a discrete and known spot on a solid support.

Another embodiment of the invention has at least one polynucleotide probe complementary to, or a fragment of, any portion of a sequence identified in the Cynomolgus monkey as a homolog of a human Tox gene, as well as at least one polynucleotide probe complementary to, or a fragment of, any portion of a Rhesus monkey gene and at least one polynucleotide probe complementary to, or a fragment of, any portion of any human gene, immobilized to a discrete and known spot on a solid support. In another embodiment of the invention, the nucleotide array has at least one polynucleotide probe that is complementary to, or a fragment of, any portion of a homolog of a human Tox gene identified in the Cynomolgus monkey, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is complementary to, or a fragment of, any portion of any of SEQ ID NOS. 43450-48714, immobilized to a discrete and known spot on a solid support. In yet another embodiment of the invention, the nucleotide array has at least one polynucleotide probe that is complementary to, or a fragment of, any portion of a homolog of a human Tox gene identified in the Cynomolgus monkey, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is any of SEQ ID NOS. 43226-48714, immobilized to a discrete and known spot on a solid support. In still yet another embodiment of the invention, the nucleotide array has at least one polynucleotide probe that is complementary to, or a fragment of, any portion of a homolog of a human Tox gene identified in the Cynomolgus monkey, where at least one polynucleotide probe from a Rhesus monkey gene is complementary to, or a fragment of, any portion of any of SEQ ID NOS. 18599-20526 and at least one polynucleotide probe from the human gene is complementary to, or a fragment of, any portion of any of SEQ ID NOS. 43450-48714, immobilized to a discrete and known spot on a solid support. In one embodiment of the invention, the nucleotide array has at least one polynucleotide probe that is complementary to, or a fragment of, any portion of a homolog of a human Tox gene identified in the Cynomolgus monkey, where at least one polynucleotide probe from a Rhesus monkey gene is complementary to, or a fragment of, any portion of any of SEQ ID NOS. 18599-20526 and at least one polynucleotide probe from the human gene is any of SEQ ID NOS. 43226-48714, immobilized to a discrete and known spot on a solid support. In another embodiment of the invention, the nucleotide array has at least one polynucleotide probe that is complementary to, or a fragment of, any portion of a homolog of a human Tox gene identified in the Cynomolgus monkey, where at least one polynucleotide probe from a Rhesus monkey gene is any of SEQ ID NOS. 35841-36074 and at least one polynucleotide probe from the human gene is complementary to, or a fragment of, any portion of any of SEQ ID NOS. 43450-48714, immobilized to a discrete and known spot on a solid support. In yet another embodiment of the invention, the nucleotide array has at least one polynucleotide probe that is complementary to, or a fragment of, any portion of a homolog of a human Tox gene identified in the Cynomolgus monkey, where at least one polynucleotide probe from a Rhesus monkey gene is any of SEQ ID NOS. 35841-36074 and at least one polynucleotide probe from the human gene is any of SEQ ID NOS. 43226-48714, immobilized to a discrete and known spot on a solid support.

Yet another embodiment of the invention has at least one polynucleotide probe complementary to, or a fragment of, any portion of any of the Cynomolgus monkey genes identified as SEQ ID NOS. 1-8881 or SEQ ID NOS. 9187-18598, as well as at least one polynucleotide probe complementary to, or a fragment of, any portion of a gene from a Rhesus monkey and at least one polynucleotide probe complementary to, or a fragment of, any portion of a human gene, immobilized to a discrete and known spot on a solid support. In one embodiment of the invention, the nucleotide array has at least one polynucleotide probe that is complementary to, or a fragment of, any portion of any of the Cynomolgus monkey genes identified as SEQ ID NOS. 1-8881 or SEQ ID NOS. 9187-18598, where at least one polynucleotide probe from a Rhesus monkey gene is complementary to, or a fragment of, any portion of any of SEQ. ID NOS. 18599-35840 or SEQ ID NOS. 36075-43225 and at least one polynucleotide probe from the human gene is complementary to, or a fragment of, any portion of any of SEQ ID NOS. 43450-48714, immobilized to a discrete and known spot on a solid support. In another embodiment of the invention, the nucleotide array has at least one polynucleotide probe that is complementary to, or a fragment of, any portion of any of the Cynomolgus monkey genes identified as SEQ ID NOS. 1-8881 or SEQ ID NOS. 9187-18598, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is any of SEQ ID NOS. 43226-48714, immobilized to a discrete and known spot on a solid support. In yet another embodiment of the invention, the nucleotide array has at least one polynucleotide probe that is complementary to, or a fragment of, any portion of any of the Cynomolgus monkey genes identified as SEQ ID NOS. 1-8881 or SEQ ID NOS. 9187-18598, where at least one polynucleotide probe from a Rhesus monkey gene is complementary to, or a fragment of, any portion of any of SEQ ID NOS. 18599-20526 and at least one polynucleotide probe from the human gene is complementary to, or a fragment of, any portion of any of SEQ ID NOS. 43450-48714, immobilized to a discrete and known spot on a solid support. In still yet embodiment of the invention, the nucleotide array has at least one polynucleotide probe that is complementary to, or a fragment of, any portion of any of the Cynomolgus monkey genes identified as SEQ ID NOS. 1-8881 or SEQ ID NOS. 9187-18598, where at least one polynucleotide probe from a Rhesus monkey gene is complementary to, or a fragment of, any portion of any of SEQ ID NOS. 18599-20526 and at least one polynucleotide probe from the human gene is any of SEQ ID NOS. 43226-48714, immobilized to a discrete and known spot on a solid support. In an embodiment of the invention, the nucleotide array has at least one polynucleotide probe that is complementary to, or a fragment of, any portion of any of the Cynomolgus monkey genes identified as SEQ ID NOS. 1-8881 or SEQ ID NOS. 9187-18598, where at least one polynucleotide probe from a Rhesus monkey gene is any of SEQ ID NOS. 35841-36074 and at least one polynucleotide probe from the human gene is complementary to, or a fragment of, any portion of any of SEQ ID NOS. 43450-48714, immobilized to a discrete and known spot on a solid support. In another embodiment of the invention, the nucleotide array has at least one polynucleotide probe that is complementary to, or a fragment of, any portion of any of the Cynomolgus monkey genes identified as SEQ ID NOS. 1-8881 or SEQ ID NOS. 9187-18598, where at least one polynucleotide probe from a Rhesus monkey gene is any of SEQ ID NOS. 35841-36074 and at least one polynucleotide probe from the human gene is any of SEQ ID NOS. 43226-48714, immobilized to a discrete and known spot on a solid support.

Still yet another embodiment of the invention has at least one polynucleotide probe complementary to, or a fragment of, any portion of any of the Cynomolgus monkey genes identified as SEQ ID NOS. 9187-18598, as well as at least one polynucleotide probe complementary to, or a fragment of, any portion of a gene from a Rhesus monkey and at least one polynucleotide probe complementary to, or a fragment of, any portion of a human gene, immobilized to a discrete and known spot on a solid support. In one embodiment of the invention, the nucleotide array has at least one polynucleotide probe that is complementary to, or a fragment of, any portion of any of the Cynomolgus monkey genes identified as SEQ ID NOS. 9187-18598, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is complementary to, or a fragment of, any portion of any of SEQ ID NOS. 43450-48714, immobilized to a discrete and known spot on a solid support. In another embodiment of the invention, the nucleotide array has at least one polynucleotide probe that is complementary to, or a fragment of, any portion of any of the Cynomolgus monkey genes identified as SEQ ID NOS. 9187-18598, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is any of SEQ ID NOS. 43226-48714, immobilized to a discrete and known spot on a solid support. In yet another embodiment of the invention, the nucleotide array has at least one polynucleotide probe that is complementary to, or a fragment of, any portion of any of the Cynomolgus monkey genes identified as SEQ ID NOS. 9187-18598, where at least one polynucleotide probe from a Rhesus monkey gene is complementary to, or a fragment of, any portion of any of SEQ ID NOS. 18599-20526 and at least one polynucleotide probe from the human gene is complementary to, or a fragment of, any portion of any of SEQ ID NOS. 43450-48714, immobilized to a discrete and known spot on a solid support. In still yet embodiment of the invention, the nucleotide array has at least one polynucleotide probe that is complementary to, or a fragment of, any portion of any of the Cynomolgus monkey genes identified as SEQ ID NOS. 9187-18598, where at least one polynucleotide probe from a Rhesus monkey gene is complementary to, or a fragment of, any portion of any of SEQ ID NOS. 18599-20526 and at least one polynucleotide probe from the human gene is any of SEQ ID NOS. 43226-48714, immobilized to a discrete and known spot on a solid support. In an embodiment of the invention, the nucleotide array has at least one polynucleotide probe that is complementary to, or a fragment of, any portion of any of the Cynomolgus monkey genes identified as SEQ ID NOS. 9187-18598, where at least one polynucleotide probe from a Rhesus monkey gene is any of SEQ ID NOS. 35841-36074 and at least one polynucleotide probe from the human gene is complementary to, or a fragment of, any portion of any of SEQ ID NOS. 43450-48714, immobilized to a discrete and known spot on a solid support. In another embodiment of the invention, the nucleotide array has at least one polynucleotide probe that is complementary to, or a fragment of, any portion of any of the Cynomolgus monkey genes identified as SEQ ID NOS. 9187-18598, where at least one polynucleotide probe from a Rhesus monkey gene is any of SEQ ID NOS. 35841-36074 and at least one polynucleotide probe from the human gene is any of SEQ ID NOS. 43226-48714, immobilized to a discrete and known spot on a solid support.

Another embodiment of the invention has at least one polynucleotide probe complementary to, or a fragment of, any portion of any of the Cynomolgus monkey genes identified as SEQ ID NOS. 17249-18598, as well as at least one polynucleotide probe complementary to, or a fragment of, any portion of a gene from a Rhesus monkey and at least one polynucleotide probe complementary to, or a fragment of, any portion of a human gene, immobilized to a discrete and known spot on a solid support. In one embodiment of the invention, the nucleotide array has at least one polynucleotide probe that is complementary to, or a fragment of, any portion of any of the Cynomolgus monkey genes identified as SEQ ID NOS. 17249-18598, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is complementary to, or a fragment of, any portion of any of SEQ ID NOS. 43450-48714, immobilized to a discrete and known spot on a solid support. In another embodiment of the invention, the nucleotide array has at least one polynucleotide probe that is complementary to, or a fragment of, any portion of any of the Cynomolgus monkey genes identified as SEQ ID NOS. 17249-18598, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is any of SEQ ID NOS. 43226-48714, immobilized to a discrete and known spot on a solid support. In yet another embodiment of the invention, the nucleotide array has at least one polynucleotide probe that is complementary to, or a fragment of, any portion of any of the Cynomolgus monkey genes identified as SEQ ID NOS. 17249-18598, where at least one polynucleotide probe from a Rhesus monkey gene is complementary to, or a fragment of, any portion of any of SEQ ID NOS. 18599-20526 and at least one polynucleotide probe from the human gene is complementary to, or a fragment of, any portion of any of SEQ ID NOS. 43450-48714, immobilized to a discrete and known spot on a solid support. In still yet embodiment of the invention, the nucleotide array has at least one polynucleotide probe that is complementary to, or a fragment of, any portion of any of the Cynomolgus monkey genes identified as SEQ ID NOS. 17249-18598, where at least one polynucleotide probe from a Rhesus monkey gene is complementary to, or a fragment of, any portion of any of SEQ ID NOS. 18599-20526 and at least one polynucleotide probe from the human gene is any of SEQ ID NOS. 43226-48714, immobilized to a discrete and known spot on a solid support. In an embodiment of the invention, the nucleotide array has at least one polynucleotide probe that is complementary to, or a fragment of, any portion of any of the Cynomolgus monkey genes identified as SEQ ID NOS. 17249-18598, where at least one polynucleotide probe from a Rhesus monkey gene is any of SEQ ID NOS. 35841-36074 and at least one polynucleotide probe from the human gene is complementary to, or a fragment of, any portion of any of SEQ ID NOS. 43450-48714, immobilized to a discrete and known spot on a solid support. In another embodiment of the invention, the nucleotide array has at least one polynucleotide probe that is complementary to, or a fragment of, any portion of any of the Cynomolgus monkey genes identified as SEQ ID NOS. 17249-18598, where at least one polynucleotide probe from a Rhesus monkey gene is any of SEQ ID NOS. 35841-36074 and at least one polynucleotide probe from the human gene is any of SEQ ID NOS. 43226-48714, immobilized to a discrete and known spot on a solid support.

Yet another embodiment of the invention has at least one polynucleotide probe directed to a Cynomolgus monkey gene, wherein the polynucleotide probe is identified as SEQ ID NOS. 8882-9186, as well as at least one polynucleotide probe complementary to, or a fragment of, any portion of a gene from a Rhesus monkey and at least one polynucleotide probe complementary to, or a fragment of, any portion of a human gene, immobilized to a discrete and known spot on a solid support. In one embodiment of the invention, the nucleotide array has at least one polynucleotide probe directed to a Cynomolgus monkey gene, wherein the polynucleotide probe is identified as SEQ ID NOS. 8882-9186, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is complementary to, or a fragment of, any portion of any of SEQ ID NOS. 43450-48714, immobilized to a discrete and known spot on a solid support. In another embodiment of the invention, the nucleotide array has at least one polynucleotide probe directed to a Cynomolgus monkey gene, wherein the polynucleotide probe is identified as SEQ ID NOS. 8882-9186, where at least one polynucleotide probe from a Rhesus monkey gene is complementary to, or a fragment of, any portion of any of SEQ ID NOS. 18599-35840 or SEQ ID NOS. 36075-43225 and at least one polynucleotide probe from the human gene is any of SEQ ID NOS. 43226-48714, immobilized to a discrete and known spot on a solid support. In yet another embodiment of the invention, the nucleotide array has at least one polynucleotide probe directed to a Cynomolgus monkey gene, wherein the polynucleotide probe is identified as SEQ ID NOS. 8882-9186, where at least one polynucleotide probe from a Rhesus monkey gene is complementary to, or a fragment of, any portion of any of SEQ ID NOS. 18599-20526 and at least one polynucleotide probe from the human gene is complementary to, or a fragment of, any portion of any of SEQ ID NOS. 43450-48714, immobilized to a discrete and known spot on a solid support. In still yet embodiment of the invention, the nucleotide array has at least one polynucleotide probe directed to a Cynomolgus monkey gene, wherein the polynucleotide probe is identified as SEQ ID NOS. 8882-9186, where at least one polynucleotide probe from a Rhesus monkey gene is complementary to, or a fragment of, any portion of any of SEQ ID NOS. 18599-20526 and at least one polynucleotide probe from the human gene is any of SEQ ID NOS. 43226-48714, immobilized to a discrete and known spot on a solid support. In an embodiment of the invention, the nucleotide array has at least one polynucleotide probe directed to a Cynomolgus monkey gene, wherein the polynucleotide probe is identified as SEQ ID NOS. 8882-9186, where at least one polynucleotide probe from a Rhesus monkey gene is any of SEQ ID NOS. 35841-36074 and at least one polynucleotide probe from the human gene is complementary to, or a fragment of, any portion of any of SEQ ID NOS. 43450-48714, immobilized to a discrete and known spot on a solid support. In another embodiment of the invention, the nucleotide array has at least one polynucleotide probe directed to a Cynomolgus monkey gene, wherein the polynucleotide probe is identified as SEQ ID NOS. 8882-9186, where at least one polynucleotide probe from a Rhesus monkey gene is any of SEQ ID NOS. 35841-36074 and at least one polynucleotide probe from the human gene is any of SEQ ID NOS. 43226-48714, immobilized to a discrete and known spot on a solid support.

As will be appreciated by a person of ordinary skill in the art, the number of polynucleotide probes immobilized to a nucleotide array will depend upon the size and composition of the nucleotide array. The polynucleotide probes of the nucleotide array may be attached to a silicon or a glass substrate. The silicon or glass substrate may have the thickness of a microscope slide or glass cover slip. Substrates that are transparent to light are useful when the assay involves optical detection. Other useful substrates include Langmuir Blodgett film, germanium, (poly)tetrafluorethylene, polystyrene, (poly)vinylidenedifluoride, polycarbonate, gallium arsenide, gallium phosphide, silicon oxide, silicon dioxide, silicon nitride, and combinations thereof. In one embodiment, the substrate is a flat glass or single crystal silicon surface with relief features less than about 10 Angstroms. In another embodiment, the substrate is a quartz wafer.

The surfaces on the substrates, to which the polynucleotide probes are attached, will usually, but not always, be composed of the same material as the substrate. Thus, the surface may comprise any number of materials, including polymers, plastics, resins, polysaccharides, silica or silica based materials, carbon, metals, inorganic glasses, membranes, silanes, or any of the above-listed substrate materials. In one embodiment, the surface will contain reactive groups, such as carboxyl, amino, and hydroxyl. In another embodiment, the surface is optically transparent and will have surface Si—OH functionalities such as are found on silica surfaces. In still yet another embodiment, the surface is silane.

In an embodiment wherein polynucleotide probes are immobilized on the substrate surface, the number of nucleic acid sequences may be selected for different applications, and may be, for example, about 100 or more, or, e.g., in some embodiments, more than 10⁵ or 10⁸. In one embodiment, the surface comprises at least 100 polynucleotide probes, each optionally having a different sequence, each polynucleotide probe contained in an area of less than about 0.1 cm², or, for example, between about 1 μm² and 10,000 μm², and each polynucleotide probe having a defined sequence and location on the substrate surface. In one embodiment, at least 1,000 different polynucleotide probes are provided on the substrate surface, wherein each nucleic acid is contained within an area less than about 10⁻³ cm², as described, for example, in U.S. Pat. No. 5,510,270, the disclosure of which is incorporated herein.

Nucleotide arrays for use in gene expression monitoring are described in PCT publication WO 97/10365, the disclosure of which is incorporated herein. In one embodiment, the polynucleotide probes are immobilized on a substrate surface, wherein the nucleotide array comprises more than 100 different polynucleotide probes and wherein each different polynucleotide probe is localized in a predetermined area of the substrate surface, and the density of the different polynucleotide probes is greater than about 60 different polynucleotide probes per 1 cm².

Nucleotide arrays containing polynucleotide probes immobilized on a surface which may be used are described in detail in U.S. Pat. No. 5,744,305, the disclosure of which is incorporated herein. As disclosed therein, on a substrate, polynucleotide probes with different sequences are immobilized each in a predefined area on a surface. For example, 10, 50, 60, 100, 10³, 10⁴, 10⁵, 10⁶, 10⁷, or 10⁸ different polynucleotide probes may be provided on the substrate surface. The polynucleotide probes of a particular sequence are provided within a predefined region of a substrate, having a surface area, for example, of about 1 cm² to 10⁻¹⁰ cm². In some embodiments, the regions have areas of less than about 10⁻¹, 10⁻², 10⁻³, 10⁻⁴, 10⁻⁵, 10⁻⁶, 10⁻⁷, 10⁻⁸, 10⁻⁹, or 10⁻¹⁰ cm². For example, in one embodiment, there is provided a planar, non-porous substrate having at least a first surface, and a number of different polynucleotide probes attached to the first surface at a density exceeding about 400 different polynucleotide probes/cm², wherein each of the different polynucleotide probes is attached to the surface of the substrate in a different predefined region, has a different determinable sequence, and is, for example, at least 4 nucleotides in length.

The polynucleotide probes may be attached to the substrate surface either by de novo synthesis on the substrate surface or by spotting or transporting polynucleotide probes onto specific locations of the substrate surface.

One example of de novo synthesis is the light-directed combinatorial synthesis of polynucleotide probes on a glass surface using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired polynucleotide probes have been synthesized on the solid surface. Combinatorial synthesis of different polynucleotide probes at different locations on the nucleotide array is determined by the pattern of illumination during synthesis and the order of addition of coupling reagents.

In addition to the foregoing, additional methods which can be used to generate a nucleotide array on a single substrate are described in PCT Publication No. WO 93/09668. In the methods disclosed in this application, reagents are delivered to the substrate by either (1) flowing within a channel defined on predefined regions or (2) “spotting” on predefined regions. However, other approaches, as well as combinations of spotting and flowing, may be employed. In each instance, certain activated regions of the substrate are mechanically separated from other regions when the monomer solutions are delivered to the various reaction sites.

A typical “flow channel” method applied to the polynucleotide probes can generally be described as follows. Diverse polymer sequences are synthesized at selected regions of a substrate or solid support by forming flow channels on a surface of the substrate through which appropriate reagents flow or in which appropriate reagents are placed. For example, assume a monomer “A” is to be bound to the substrate in a first group of selected regions. If necessary, all or part of the surface of the substrate in all or a part of the selected regions is activated for binding by, for example, flowing appropriate reagents through all or some of the channels, or by washing the entire substrate with appropriate reagents. After placement of a channel block on the surface of the substrate, a reagent having the monomer A flows through or is placed in all or some of the channel(s). The channels provide fluid contact to the first selected regions, thereby binding the monomer A on the substrate directly or indirectly (via a spacer) in the first selected regions.

Thereafter, a monomer B is coupled to second selected regions, some of which may be included among the first selected regions. The second selected regions will be in fluid contact with a second flow channel(s) through translation, rotation, or replacement of the channel block on the surface of the substrate; through opening or closing a selected valve; or through deposition of a layer of chemical or photoresist. If necessary, a step is performed for activating at least the second regions. Thereafter, the monomer B is flowed through or placed in the second flow channel(s), binding monomer B at the second selected locations. In this particular example, the resulting sequences bound to the substrate at this stage of processing will be, for example, A, B, and AB. The process is repeated to form a polynucleotide probe of desired length at known locations on the substrate.

After the substrate is activated, monomer A can be flowed through some of the channels, monomer B can be flowed through other channels, a monomer C can be flowed through still other channels, etc. In this manner, many or all of the reaction regions are reacted with a monomer before the channel block must be moved or the substrate must be washed and/or reactivated. By making use of many or all of the available reaction regions simultaneously, the number of washing and activation steps can be minimized.

A person of ordinary skill in the art will recognize that there are alternative methods of forming channels or otherwise protecting a portion of the surface of the substrate. For example, according to some embodiments, a protective coating such as a hydrophilic or hydrophobic coating (depending upon the nature of the solvent) is utilized over portions of the substrate to be protected, sometimes in combination with materials that facilitate wetting by the reactant solution in other regions. In this manner, the flowing solutions are further prevented from passing outside of their designated flow paths.

The “spotting” methods of preparing polynucleotide probes can be implemented in much the same manner as the flow channel methods. For example, a monomer A can be delivered to and coupled with a first group of reaction regions which have been appropriately activated. Thereafter, a monomer B can be delivered to and reacted with a second group of activated reaction regions. Unlike the flow channel embodiments described above, reactants are delivered by directly depositing (rather than flowing) relatively small quantities of them in selected regions. In some steps, of course, the entire substrate surface can be sprayed or otherwise coated with a solution. In preferred embodiments, a dispenser moves from region to region, depositing only as much monomer as necessary at each stop. Typical dispensers include a micropipette to deliver the monomer solution to the substrate and a robotic system to control the position of the micropipette with respect to the substrate. In other embodiments, the dispenser includes a series of tubes, a manifold, a nucleotide array of pipettes, or the like so that various reagents can be delivered to the reaction regions simultaneously.

Furthermore, other methods or materials may be used to attached the polynucleotide probes to the substrate surface, such as using a polymer including a substantial amount of monomer or monomers including uncharged polar moieties other than primary amide, such as a polymer including an N-substituted acrylamide, N,N-disubstituted acrylamide, N-substituted methacrylamide, and/or N,N-disubstituted methacrylamide or coating the surface of the material with thermochemically reactive groups. See U.S. Publication Nos. 2005/0074478; 2003/0078314; 2001/0055761; and 2001/0014448.

In one embodiment, the polynucleotide probes are attached to the substrate surface. In this embodiment, a linker molecule, attached to a silane matrix, provides a surface that may be spatially activated by light. In this embodiment, synthesis of the polynucleotide probes occurs in parallel, such that the addition of a nucleotide to multiple growing polynucleotide probe chains occurs simultaneously.

In this embodiment, to define which polynucleotide probe chains will receive a nucleotide in each step, photolithographic masks, carrying 18-20 square micron windows that correspond to the dimension of individual features, may be placed over the substrate. The windows are distributed over the mask based on the desired sequence of each polynucleotide probe. When ultraviolet light is shone over the mask in the first step of synthesis, the exposed linkers become deprotected and are available for nucleotide coupling.

Additionally, a person of ordinary skill in the art will know that the number of polynucleotide probes immobilized to a nucleotide array will depend upon the end use of the nucleotide array. For certain diagnostic nucleotide arrays, only a few different polynucleotide probes may be required. However, other uses of the nucleotide array, such as for analyzing the transcriptional state of a cell or tissue in response to a therapeutic agent, a large number of polynucleotide probes immobilized to a solid support may be required to collect the desired information.

In one embodiment, the nucleotide array consists of polynucleotide probes complementary to, or a fragment of, any portion of any of SEQ ID NOS. 1-8881 or SEQ ID NOS. 9187-18598 immobilized to the solid support. Optionally, the nucleotide array consists of polynucleotide probes complementary to, or a fragment of, any portion of any of SEQ ID NOS. 1-8881 or SEQ ID NOS. 9187-18598, as well as polynucleotide probes complementary to, or a fragment of, any portion of any of SEQ ID NOS. 18599-35840 or SEQ ID NOS. 36075-43225 and/or SEQ ID NOS. 43450-48714 immobilized to the solid support.

In another embodiment, the nucleotide array consists of polynucleotide probes complementary to, or fragments of, any portion of each of SEQ ID NOS. 1-8881 and SEQ ID NOS. 9187-18598 immobilized to the solid support. Optionally, the nucleotide array consists of polynucleotide probes complementary to, or fragments of, any portion of each of SEQ ID NOS. 1-8881 and SEQ ID NOS. 9187-18598, as well as polynucleotide probes complementary to, or fragments of, any portion of each of SEQ ID NOS. 18599-35840 or SEQ ID NOS. 36075-43225 and/or SEQ ID NOS. 43450-48714 immobilized to the solid support.

In yet another embodiment, subsets of polynucleotide probes complementary to, or fragments of, any portion of any of SEQ ID NOS. 1-8881 and SEQ ID NOS. 9187-18598 are immobilized to the solid support. These subsets may include the polynucleotide probes that are complementary to, or fragments of, any portion of the Cynomolgus monkey genes that are homologous to the human Tox genes. Optionally, the subsets of polynucleotide probes may include polynucleotide probes that are complementary to, or fragments of, any portion of any of the Cynomolgus and Rhesus monkey genes that are homologous to the human Tox genes. As a non-limiting example, the subsets may include the polynucleotide probes that are complementary to, or fragments of, any portion of any of SEQ ID NOS. 17249-18598. Optionally, as a non-limiting example, the subsets may include the polynucleotide probes that are complementary to, or fragments of, any portion of any of SEQ ID NOS. 17249-18598 and any of SEQ ID NOS. 18599-20526. Further, optionally, as a non-limiting example, the subsets may include the polynucleotide probes that are complementary to, or fragments of, any portion of any of SEQ ID NOS. 17249-18598, as well as any of SEQ ID NOS. 18599-20526 and/or SEQ ID NOS. 43450-48714.

In still yet another embodiment, the subsets of polynucleotide probes complementary to, or fragments of, any portion of any of SEQ ID NOS. 1-8881 and SEQ ID NOS. 9187-18598 may include the polynucleotide probes that are complementary to, or fragments of, any of the Cynomolgus monkey genes that are orthologs of known human genes. Optionally, the subsets of polynucleotide probes may include the polynucleotide probes that are complementary to, or fragments of, any of the Cynomolgus and Rhesus monkey genes that are orthologs of known human genes. As a non-limiting example, the subsets may include the polynucleotide probes that are complementary to, or fragments of, any portion of any of SEQ ID NOS. 9187-18598. Optionally, as a non-limiting example, the subsets may include the polynucleotide probes that are complementary to, or fragments of, any portion of any of SEQ ID NOS. 9187-18598 and any of SEQ ID NOS. 18599-35840 or SEQ ID NOS. 36075-43225. Further, optionally, as a non-limiting example, the subsets may include the polynucleotide probes that are complementary to, or fragments of, any portion of any of SEQ ID NOS. 9187-18598, as well as any of SEQ ID NOS. 18599-35840 or SEQ ID NOS. 36075-43225 and/or SEQ ID NOS. 43450-48714.

In another embodiment, the nucleotide array consists of polynucleotide probes of any of SEQ ID NOS. 8882-9186 immobilized to the solid support. Optionally, the nucleotide array consists of polynucleotide probes of any of SEQ ID NOS. 8882-9186, as well as polynucleotide probes of any of SEQ ID NOS. 35841-36074 and/or SEQ ID NOS. 43450-48714 immobilized to the solid support.

In another embodiment, the subsets may include about 100 to about 55,000 polynucleotide probes. Alternatively, the subsets may include about 500 to about 50,000 polynucleotide probes. In one embodiment, the subsets may include about 1000 to about 45,000 polynucleotide probes. In another embodiment, the subsets may include about 2500 to about 40,000 polynucleotide probes. In yet another embodiment, the subsets may include 5000 to about 35,000 polynucleotide probes. In still yet another embodiment, the subsets may include 10,000 to about 30,000 polynucleotide probes. In another embodiment, the subsets may include 15,000 to about 25,000 polynucleotide probes.

The polynucleotide probes complementary to, or fragments of, any portion of any of SEQ ID NOS. 1-8881, SEQ ID NOS. 9187-18598, SEQ ID NOS. 18599-35840 or SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449 were designed to be complementary to one or more selected, known target nucleic acid sequences. These polynucleotide probes are designed to hybridize either to the target nucleic acid sequence itself or to variants of the target nucleic acid sequence. The variants of the target nucleic acid sequence may differ from the target nucleic acid sequence at one or more positions, but show a high overall degree of sequence identity with the reference sequence (e.g., at least 75, 90, 95, 99, 99.9 or 99.99%). The degree of identity between a base region of a polynucleotide probe and a base region of a target nucleic acid sequence can be determined by manual alignment. The degree of identity is determined by comparing just the sequence of nitrogenous bases, irrespective of the sugar and backbone regions of the nucleic acids being compared. Thus, the polynucleotide probe:target nucleic acid base sequence alignment may be DNA:DNA, RNA:RNA, DNA:RNA, RNA:DNA, or any combinations or analogs thereof. Equivalent RNA and DNA base sequences can be compared by converting U's (in RNA) to T's (in DNA).

2) Normalization Controls

Normalization controls are polynucleotide probes that have a sequence deliberately selected to be perfectly complementary to, or fragments of, labeled target polynucleotide probes added to the test or control sample either before or after the RNA, such as mRNA, of the test or control samples is amplified. The signals obtained from the normalization controls after hybridization provide a control for variations in hybridization conditions, label intensity, “reading” efficiency and other factors that may cause the signal of a perfect hybridization to vary between nucleotide arrays. In one embodiment, signals (e.g., fluorescence intensity) read from all other polynucleotide probes in the nucleotide array are divided by the signal (e.g., fluorescence intensity) from the normalization controls thereby normalizing the measurements.

Virtually any polynucleotide probe may serve as a normalization control. However, it is recognized that hybridization efficiency varies with base composition and polynucleotide probe length. Preferred normalization polynucleotide probes are selected to reflect the average length of the other polynucleotide probes present in the nucleotide array, however, they can be selected to cover a range of lengths. The normalization control(s) can also be selected to reflect the (average) base composition of the other polynucleotide probes in the nucleotide array, however in one embodiment, only one or a few normalization controls are used and they are selected such that they hybridize well (i.e. no secondary structure) and do not match any target polynucleotide probes generated from the test or control samples.

Normalization controls may be localized at any position in the nucleotide array or at multiple positions throughout the nucleotide array to control for spatial variation in hybridization efficiently. In one embodiment, the normalization controls are located at the corners or at the edges of the nucleotide array, as well as in the middle.

3) Expression Level Controls

Expression level controls are polynucleotide probes that have a sequence deliberately selected to be perfectly complementary to, or fragments of, constitutively expressed genes in the biological sample. Expression level controls are designed to control for the overall health and metabolic activity of a cell. Examination of the covariance of an expression level control with the expression level of the target nucleic acid indicates whether measured changes or variations in expression level of a gene are due to changes in transcription rate of that gene or to general variations in health of the cell. Thus, for example, when a cell is in poor health or lacking a critical metabolite the expression levels of both an active gene and a constitutively expressed gene are expected to decrease. The converse is also true. Thus, where the expression levels of both an expression level control and the gene appear to both decrease or to both increase, the change may be attributed to changes in the metabolic activity of the cell as a whole, not to differential expression of the gene in question. Conversely, where the expression levels of the gene and the expression level control do not covary, the variation in the expression level of the target gene is attributed to differences in regulation of that gene and not to overall variations in the metabolic activity of the cell.

Virtually any constitutively expressed gene provides a suitable target for expression level controls. Typically, expression level control polynucleotide probes have sequences complementary to, or fragments of, subsequences of constitutively expressed “housekeeping genes” including, but not limited to the β-actin gene, the transferrin receptor gene, or the GAPDH gene.

4) Mismatch Controls

Mismatch controls may also be provided for the polynucleotide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449, for expression level controls, or for normalization controls. Mismatch controls are polynucleotide probes that have a sequence deliberately selected not to be perfectly complementary to a particular target nucleic acid. The mismatch control typically has a corresponding test polynucleotide probe that is perfectly complementary to the sequence of the same particular target nucleic acid except for the presence of one or more mismatched bases. A mismatched base is a base selected so that it is not complementary to, or fragment of, the corresponding base in the sequence of the target nucleic acid to which the nucleic acid probe would otherwise specifically hybridize. One or more mismatches are selected such that under appropriate hybridization conditions (e.g. stringent conditions), the polynucleotide probe complementary to, or a fragment of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449 would be expected to hybridize with its target nucleic acid, but the mismatch control would not hybridize (or would hybridize to a significantly lesser extent).

In one embodiment, the mismatch control would contain a central mismatch. Thus, for example, where a polynucleotide probe contains 20 nucleotides, a corresponding mismatch control will have the identical sequence except for a single base mismatch (e.g., substituting a G, a C or a T for an A) at any of positions 6 through 14 (the central mismatch).

Mismatch controls thus provide a control for non-specific binding or cross-hybridization to a nucleic acid in the sample other than the target to which the polynucleotide probe is directed. Mismatch controls thus indicate whether a hybridization is specific or not. For example, if the target is present, the perfect match polynucleotide probes should be consistently brighter than the mismatch controls. In addition, if all central mismatches are present, the mismatch polynucleotide probes can be used to detect a mutation, such as a SNP.

5) Sample Preparation/Amplification Controls

The nucleotide array may also include sample preparation/amplification controls. Sample preparation/amplification controls are polynucleotide probes that are complementary to, or fragments of, subsequences of control genes selected because the control genes do not normally occur in the nucleic acids of the particular biological sample being assayed. Suitable sample preparation/amplification controls include, for example, polynucleotide probes to bacterial genes (e.g., Bio B).

The sample preparation/amplification control may be added to the test or control sample before processing. Quantification of the hybridization of the sample preparation/amplification control polynucleotide probe provides a measure of alteration in the abundance of the nucleic acids caused by the subsequent processing steps (e.g. PCR, reverse transcription, in vitro transcription, etc.).

Test or Control Sample

The test sample may be either total cellular RNA, e.g., mRNA, directly isolated from, or a target nucleic acid complementary to, the RNA isolated from, a biological sample obtained from a primate, e.g., a human, a Cynomolgus monkey, or a Rhesus monkey, especially a non-human primate such as a Cynomolgus monkey or a Rhesus monkey, exposed to the therapeutic agent. Alternatively, the test sample may be either total RNA, e.g., mRNA, directly isolated from, or a nucleic acid complementary to the RNA isolated from, a biological sample obtained from a primate, e.g., a human, a Cynomolgus monkey, or a Rhesus monkey, especially a non-human primate such as a Cynomolgus monkey or a Rhesus monkey, that has a SNP. The test sample includes, but is not limited to isolated RNA, a cDNA reverse transcribed from the isolated RNA, an RNA transcribed from the cDNA, a DNA amplified from the cDNA, and an RNA transcribed from the amplified DNA.

The control sample may be either total cellular RNA, e.g., mRNA, directly isolated from, or a nucleic acid complementary to the RNA isolated from, a biological sample obtained from a primate, e.g., a human, a Cynomolgus monkey, or a Rhesus monkey, especially a non-human primate such as a Cynomolgus monkey or a Rhesus monkey, that was not exposed to the therapeutic agent. Alternatively, the control sample may be either total cellular RNA, e.g., mRNA, directly isolated from, or a nucleic acid complementary to the RNA isolated from, a biological sample obtained from a primate, e.g., a human, a Cynomolgus monkey, or a Rhesus monkey, especially a non-human primate such as a Cynomolgus monkey or a Rhesus monkey, that lacks the SNP. The control sample includes, but is not limited to isolated RNA, a cDNA reverse transcribed from the isolated RNA, an RNA transcribed from the cDNA, a DNA amplified from the cDNA, and an RNA transcribed from the amplified DNA.

The test and/or control samples may be amplified prior to hybridization to the polynucleotide probe immobilized to the solid surface of the nucleotide array. Methods of amplification are well known to persons of ordinary skill in the art. One method by which the test sample may be amplified is PCR.

In one embodiment, the test or control sample RNA is reverse transcribed with a reverse transcriptase and a promoter consisting of a sequence encoding the phage T7 promoter to provide single stranded DNA template. The DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA.

It will be appreciated by a person of ordinary skill in the art that the direct transcription method described above provides an antisense RNA pool. Where antisense RNA is used as the target nucleic acid in the test or control sample, the polynucleotide probes provided in the nucleotide array are chosen to be complementary to, or fragments of, subsequences of the antisense RNA. Conversely, where the test or control sample is a pool of sense nucleic acids, the polynucleotide probes are selected to be complementary to, or fragments of, subsequences of the sense nucleic acids. Finally, where the test or control sample is double stranded, the polynucleotide probes may be of either sense as the target nucleic acids include both sense and antisense strands.

Labeling of the Test or Control Sample

Formation of the hybridized complex between the polynucleotide probes immobilized on the solid surface of the nucleotide array and the test or control sample may be monitored by detecting one or more labels attached to the test or control sample. The labels may be incorporated by any number of means known to persons of ordinary skill in the art. In one embodiment of the present invention, the label may be incorporated using PCR. In another embodiment of the present invention, a labeled nucleotide, such as fluorosceien-labeled UTP and/or CTP, may be incorporated into transcribed nucleic acids using transcription amplification.

The means of attaching labels to nucleic acids are well known to persons of ordinary skill in the art. Examples of attachment methods include, but are not limited to, nick translation and end-labeling by kinasing the nucleic acid and subsequently attaching a nucleic acid linker joining the test or control sample to a label, such as a fluorophore.

Additionally, a label may be added directly to the test or control sample if the test or control sample is RNA directly isolated from the Cynomolgus or Rhesus monkey biological sample. Furthermore, a label may be added directly to the amplification product after the amplification is completed.

Labels suitable for use in the present invention include any composition detectable by spectroscopic, photochemical, biochemical, immunochemical, electrical, optical, or chemical means. The label may be any suitable labeling substance, including but not limited to a radioisotope, an enzyme, an enzyme cofactor, an enzyme substrate, a dye, a hapten, a chemiluminescent molecule, a fluorescent molecule, a phosphorescent molecule, an electrochemiluminescent molecule, a chromophore, a base sequence region that is unable to stably hybridize to the target nucleic acid under the stated conditions, and mixtures of these. Useful labels in the present invention include biotin for staining with labeled streptavidin conjugate, magnetic beads, such as Dynabeads™, fluorescent dyes, such as fluoroscein, Texas red, rhodamine, and green fluorescent protein, radiolabels, such as ³H, ¹²⁵I, ³⁵S, ¹⁴C, and ³²P, enzymes, such as horse radish peroxidase and alkaline phosphatase, and calorimetric labels, such as colloidal gold and colored glass or plastic beads. In one embodiment, the label is biotin.

The labels may be detected using a variety of means known by a person of ordinary skill in the art. Radiolabels may be detected using photographic film or scintillation counters. Fluorescent markers may be detected using a photodetector to detect emitted light. Enzymatic labels may be detected by providing the enzyme with a substrate and detecting the reaction product produced by the action of the enzyme on the substrate. Calorimetric labels may be detected by simply visualizing the colored label.

The label may be added to the target nucleic acid(s) of the test or control sample prior to, or after the hybridization. So called “direct labels” are detectable labels that are directly attached to or incorporated into the target (test or control sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Hybridizing the Polynucleotide Probes and the Test Sample

Nucleic acid hybridization simply involves providing a denatured polynucleotide probe and nucleic acid of the test or control sample under conditions where the polynucleotide probe and its complementary nucleic acid can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

Hybridization conditions may be selected to provide any degree of stringency. In one embodiment, hybridization reaction between the polynucleotide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449 and the test or control sample may be performed under low stringency conditions (e.g., 6×SSPE-T at 37° C. (0.005% Triton X-100)) to ensure hybridization. The hybridized complexes may subsequently be washed under higher stringency conditions (e.g., 1×SSPE-T at 37° C.) to eliminate mismatched hybridized complexes. Successive washes may be performed at increasingly higher stringency (e.g., down to as low as 0.25×SSPE-T at 37° C. to 50° C.) until a desired level of hybridization specificity is obtained. In another embodiment, the degree of stringency may also be increased by adding additional agents, such as formamide. Hybridization specificity may be evaluated by comparison of hybridization to the polynucleotide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449 with hybridization to the various control polynucleotide probes that may be present on the nucleotide array (e.g., expression level control, normalization control, mismatch control, etc.).

In general, a tradeoff exists between hybridization specificity (stringency) and signal intensity. In one embodiment, the wash is performed at the highest stringency that produces consistent results and that provides a signal intensity greater than approximately 10% of the background intensity. In another embodiment, the nucleotide array containing the hybridized complexes may be washed at successively higher stringency solutions and read between each wash. Analysis of the data sets thus produced will reveal a wash stringency above which the hybridization pattern is not appreciably altered and which provides adequate signal for the particular polynucleotide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449 of interest.

The background signal may be reduced by using a detergent, such as C-TAB, or a blocking reagent, such as sperm DNA or cot-1 DNA, during the hybridization reaction to the reduce non-specific binding of the labeled test or control sample. In one embodiment of the present invention, the hybridization reaction may be performed in the presence of about 0.5 mg/ml DNA, such as herring sperm DNA.

The stability of the hybridized complexes formed between the polynucleotide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449, are generally in the order of RNA:RNA>RNA:DNA>DNA:DNA, in solution. Long polynucleotide probes will generally from a more stable hybridized complex with the test sample. However, longer polynucleotide probes generally exhibit poorer mismatch discrimination than shorter polynucleotide probes. Mismatch discrimination refers to the measured hybridization signal ratio between a perfect match polynucleotide probe and a single base mismatch polynucleotide probe. Shorter polynucleotide probes (e.g., containing 8 nucleotides) discriminate mismatches very well, but the overall duplex stability is low.

Altering the thermal stability (“Tₘ”) of the duplex formed between the target nucleic acid and the polynucleotide probe using, e.g., known polynucleotide probe analogues allows for optimization of duplex stability and mismatch discrimination. One useful aspect of altering the Tₘ arises from the fact that adenine-thymine (A-T) duplexes have a lower Tₘ than guanine cytosine (G-C) duplexes due in part to the fact that the A-T duplexes have 2 hydrogen bonds per base-pair, while the G-C duplexes have 3 hydrogen bonds per base pair. In heterogeneous nucleotide arrays in which there is a non-uniform distribution of bases, it is not generally possible to optimize hybridization for each polynucleotide probe simultaneously. Thus, in some embodiments, it is desirable to selectively destabilize G-C duplexes and/or to increase the stability of A-T duplexes. This can be accomplished, e.g., by substituting guanine residues in the polynucleotide probes of a nucleotide array which form G-C duplexes with hypoxanthine, or by substituting adenine residues in polynucleotide probes which form A-T duplexes with 2,6 diaminopurine or by using the salt tetramethyl ammonium chloride (TMACl) in place of NaCl.

Altered duplex stability conferred by using polynucleotide probe analogues can be ascertained by following, e.g., fluorescence signal intensity of nucleotide arrays hybridized with a target nucleic acid over time. The data allow optimization of specific hybridization conditions at, e.g., room temperature (for simplified diagnostic applications in the future).

Another way of verifying altered duplex stability is by following the signal intensity generated upon hybridization with time. Previous experiments using DNA targets and DNA chips have shown that signal intensity increases with time, and that the more stable duplexes generate higher signal intensities faster than less stable duplexes. The signals reach a plateau or “saturate” after a certain amount of time due to all of the binding sites becoming occupied. These data allow for optimization of hybridization, and determination of the best conditions at a specified temperature.

Methods of optimizing hybridization conditions are well known to those of skill in the art (see, e.g., Laboratory Techniques in Biochemistry and Molecular Biology, Vol. 24: Hybridization With Nucleic Acid Probes, P. Tijssen, ed. Elsevier, N.Y., (1993)).

The hybridization signals will vary in strength with the efficiency of the hybridization of the polynucleotide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449, immobilized to the solid surface of the nucleotide array, with the test or control sample. Additionally, the hybridization signal will vary with the amount of label incorporated into the test or control sample. Further, the hybridization signal will vary in strength with the amount of the particular nucleic acid in the test or control sample.

Therefore, the nucleotide array containing a plurality of polynucleotide probes complementary to, or fragments of, any portion of any of SEQ ID NOS. 1-8881, SEQ ID NOS. 9187-18598, SEQ ID NOS. 18599-35840, SEQ iD NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449, immobilized to the solid surface, may be used to determine the levels and species of RNA produced after administration of a therapeutic agent to a primate, e.g., a human, a Cynomolgus monkey, or a Rhesus monkey, especially a non-human primate such as a Cynomolgus monkey or a Rhesus monkey. The hybridization patterns and intensities of the label attached to the test or control sample may be determined by hybridizing the test or control sample to the polynucleotide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449. The hybridization pattern produced from a test sample obtained from a biological sample of a primate, e.g., a human, a Cynomolgus monkey, or a Rhesus monkey, especially a non-human primate such as a Cynomolgus monkey or a Rhesus monkey, administered a therapeutic agent, may be compared with the hybridization pattern produced from a control sample obtained from a biological sample from the same species of primate that was not administered the therapeutic agent. The differences between the two hybridization patterns will indicate which primate genes are affected upon administration of the therapeutic agent. The investigator may then determine the genes which are up- or down-regulated to determine the biological effects, including the actions, targets, and toxicities, of the therapeutic agent.

Based upon the differences in hybridization patterns, an investigator may also determine whether a modified therapeutic agent is more specific and/or active than the originally administered therapeutic agent. If fewer primate genes are up- or down-regulated after administration of the modified therapeutic agent, as compared with the genes up- or down-regulated after administration of the original therapeutic agent, the modified therapeutic agent is more specific than the original therapeutic agent.

Modifying the Test or Control Sample to Decrease Background

The test or control sample may be modified prior to hybridization to the polynucleotide probes on the nucleotide array to reduce sample complexity and thereby decrease background signal and improve sensitivity of the measurement. In one embodiment, complexity reduction is achieved by selective degradation of background RNA. Selective degradation is accomplished by hybridizing the sample RNA (e.g., polyA⁺ RNA) with a pool of DNA oligonucleotides that hybridize specifically with the regions to which the polynucleotide probes in the nucleotide array specifically hybridize. In one embodiment, the pool of oligonucleotides consists of the same polynucleotide probes as found on the nucleotide array.

The pool of oligonucleotides hybridizes to the test or control sample RNA forming a number of double stranded (hybrid duplex) nucleic acids. The hybridized sample is then treated with RNase A, a nuclease that specifically digests single stranded RNA. The RNase A is then inhibited, using a protease and/or commercially available RNase inhibitors, and the double stranded nucleic acids are then separated from the digested single stranded RNA. This separation may be accomplished in a number of ways well known to those of ordinary skill in the art including, but not limited to, electrophoresis, and gradient centrifugation. However, in one embodiment, the pool of DNA oligonucleotides is provided attached to beads forming thereby a nucleic acid affinity column. After digestion with the RNase A, the hybridized DNA is removed simply by denaturing (e.g., by adding heat or increasing salt) the hybrid duplexes and washing the previously hybridized RNA off in an elution buffer.

The undigested RNA fragments which will be hybridized to the polynucleotide probe in the nucleotide array are then preferably end-labeled with a fluorophore attached to an RNA linker using an RNA ligase. This procedure produces a labeled sample RNA pool in which the nucleic acids that do not correspond to polynucleotide probes in the nucleotide array are eliminated and thus unavailable to contribute to a background signal.

Another method of reducing sample complexity involves hybridizing the RNA with deoxyoligonucleotides that hybridize to regions that border on either size the regions to which the polynucleotide probes of the nucleotide array are directed. Treatment with RNAse H selectively digests the double stranded (hybrid duplexes) leaving a pool of single-stranded RNA corresponding to the short regions that were formerly bounded by the deoxyolignucleotide polynucleotide probes and which correspond to the target nucleic acids and longer RNA sequences that correspond to regions between the target nucleic acids and the polynucleotide probes of the nucleotide array. The short RNA fragments are then separated from the long fragments (e.g., by electrophoresis), labeled if necessary as described above, and then are ready for hybridization with the nucleotide array.

In a third approach, sample complexity reduction involves the selective removal of particular (preselected) mRNA messages. In particular, highly expressed mRNA messages that are not specifically polynucleotide probed by the polynucleotide probes in the nucleotide array are preferably removed. This approach involves hybridizing the polyA⁺ mRNA with an oligonucleotide polynucleotide probe that specifically hybridizes to the preselected message close to the 3′ (poly A) end. The polynucleotide probe may be selected to provide high specificity and low cross reactivity. Treatment of the hybridized message/polynucleotide probe complex with RNase H digests the double stranded region effectively removing the polyA⁺ tail from the rest of the message. The sample is then treated with methods that specifically retain or amplify polyA⁺ RNA (e.g., an oligo dT column or (dT)ₙ magnetic beads). Such methods will not retain or amplify the selected message(s) as they are no longer associated with a polyA⁺ tail. These highly expressed messages are effectively removed from the sample providing a sample that has reduced background mRNA.

Signal Evaluation

A person of ordinary skill in the art will appreciate that methods for evaluating the hybridization results vary with the nature of the specific polynucleotide probe and nucleic acids used, as well as the controls (e.g., normalization controls, expression level controls, mismatch controls, or sample preparation/amplification controls) provided. In one embodiment, simple quantification of the fluorescence intensity for each polynucleotide probe is determined. This is accomplished simply by measuring polynucleotide probe signal strength at each location (representing a different polynucleotide probe) on the nucleotide array (e.g., where the label is a fluorescent label, detection of the amount of florescence (intensity) produced by a fixed excitation illumination at each location on the nucleotide array). Comparison of the absolute intensities of a nucleotide array hybridized to nucleic acids from a test or control sample with intensities produced by a hybridization to the expression level controls, mismatch controls, normalization controls, or sample preparation/amplification controls provides a measure of the relative expression of the nucleic acids that hybridize to each of the polynucleotide probes.

A person of ordinary skill in the art, however, will appreciate that hybridization signals will vary in strength with efficiency of hybridization, the amount of label on the target nucleic acid and the amount of the particular target nucleic acid in the sample. Typically target nucleic acids present at very low levels (e.g., <1 pM) will show a very weak signal. At some low level of concentration, the signal becomes virtually indistinguishable from background. In evaluating the hybridization data, a threshold intensity value may be selected below which a signal is not counted as being essentially indistinguishable from background.

Where it is desirable to detect nucleic acids expressed at lower levels, a lower threshold is chosen. Conversely, where only high expression levels are to be evaluated a higher threshold level is selected. In one embodiment, a suitable threshold is about 10% above that of the average background signal.

In addition, the provision of appropriate controls (e.g., normalization controls, expression level controls, mismatch controls, and sample preparation/amplification controls) permits a more detailed analysis that controls for variations in hybridization conditions, cell health, non-specific binding and the like. Thus, for example, in one embodiment, the nucleotide array is provided with normalization controls as described above. These normalization controls are polynucleotide probes complementary to, or fragments of, control sequences added in a known concentration to the sample. Where the overall hybridization conditions are poor, the normalization controls will show a smaller signal reflecting reduced hybridization. Conversely, where hybridization conditions are good, the normalization controls will provide a higher signal reflecting the improved hybridization. Normalization of the signal complementary to, or fragments of, other polynucleotide probes in the nucleotide array to the normalization controls thus provides a control for variations in hybridization conditions. Typically, normalization is accomplished by dividing the measured signal from the other polynucleotide probes in the nucleotide array by the average signal produced by the normalization controls. Normalization may also include correction for variations due to sample preparation and amplification. Such normalization may be accomplished by dividing the measured signal by the average signal from the sample preparation/amplification control polynucleotide probes (e.g., the Bio B polynucleotide probes). The resulting values may be multiplied by a constant value to scale the results.

As indicated above, the nucleotide array may include mismatch controls. In one embodiment, there may be a mismatch control having a central mismatch for every polynucleotide probe (except the normalization controls) in the nucleotide array. It is expected that after washing in stringent conditions, where a perfect match would be expected to hybridize to the polynucleotide probe, but not to the mismatch, the signal from the mismatch controls should only reflect non-specific binding or the presence in the sample of a nucleic acid that hybridizes with the mismatch. Where both the polynucleotide probe in question and its corresponding mismatch control both show high signals, or the mismatch shows a higher signal than its corresponding test polynucleotide probe, there is a problem with the hybridization and the signal from those polynucleotide probes may be ignored. The difference in hybridization signal intensity between the target specific polynucleotide probe and its corresponding mismatch control is a measure of the discrimination of the target-specific polynucleotide probe. Thus, in one embodiment, the signal of the mismatch polynucleotide probe is subtracted from the signal from its corresponding test polynucleotide probe to provide a measure of the signal due to specific binding of the test polynucleotide probe.

The concentration of a particular sequence can then be determined by measuring the signal intensity of each of the polynucleotide probes that bind specifically to that gene and normalizing to the normalization controls. Where the signal from the polynucleotide probes is greater than the mismatch, the mismatch is subtracted. Where the mismatch intensity is equal to or greater than its corresponding test polynucleotide probe, the signal is ignored. The expression level of a particular gene can then be scored by the number of positive signals (either absolute or above a threshold value), the intensity of the positive signals (either absolute or above a selected threshold value), or a combination of both metrics (e.g., a weighted average).

Monitoring Expression Levels

As indicated above, the methods of this invention may be used to monitor expression levels of a gene in a wide variety of contexts. For example, where the effects of a therapeutic agent on gene expression of a primate, e.g., a human, a Cynomolgus monkey, or a Rhesus monkey, is to be determined the therapeutic agent will be administered to the primate. Nucleic acids from a biological sample from the primate and from a primate not administered the therapeutic agent may be isolated, amplified, and hybridized to a nucleotide array containing polynucleotide probes directed to the gene of interest. The expression levels of that particular gene may be determined as described above. The same method may be followed when identifying the targets of a therapeutic agent in a primate, in determining the effects of a therapeutic agent on a primate, in determining whether a specific gene is a target of a therapeutic agent, and in determining whether a putative target for a therapeutic agent is an actual target for a therapeutic agent. In one embodiment of the invention, the primate is a non-human primate, e.g., a Cynomolgus monkey or a Rhesus monkey.

The present invention moreover provides a method of predicting at least one toxic effect of a compound, comprising:

(a) detecting the level of expression of one or more genes identified as SEQ ID NOS. 1-8881 and SEQ ID NOS. 9187-18598 in a biological sample exposed to the compound;

(b) comparing the level of expression of the genes to their level of expression in a control tissue or cell sample, wherein differential expression of the genes identified as orthologs of human Tox genes in the Cynomolgus monkey, indicated as SEQ ID NOS. 1-8881 and SEQ ID NOS. 9187-18598, is evidence of at least one toxic effect.

Optionally, the present invention provides a method of predicting at least one toxic effect of a compound, comprising:

(a) detecting the level of expression of one or more genes identified as SEQ ID NOS. 1-8881 and SEQ ID NOS. 9187-18598, as well as SEQ ID NOS. 18599-35840, SEQ ID NOS. 36075-43225, and SEQ ID NOS. 36075-43225 in a biological sample exposed to the compound;

(b) comparing the level of expression of the genes to their level of expression in a control biological sample, wherein differential expression of the genes identified as orthologs of human Tox genes in the Cynomolgus monkey, indicated as SEQ ID NOS. 17249-18598, as well as differential expression of the genes identified as orthologs of human Tox genes in the Rhesus monkey, indicated as SEQ ID NOS. 18599-20526, is evidence of at least one toxic effect.

Also, as indicated above, the nucleotide array of the present invention may be used to identify biomarkers upon administration of a therapeutic agent comprising administering the therapeutic agent to a primate, e.g., a human, a Cynomolgus monkey, or a Rhesus monkey, especially to a non-human primate such as a Cynomolgus monkey or a Rhesus monkey, isolating the RNA from a biological sample from the non-human primate to yield a test sample, and hybridizing the test sample with a nucleotide array containing at least one polynucleotide probe complementary to, or a fragment of, any portion of a Cynomolgus monkey gene. Optimally, the RNA from the test sample may be amplified prior to hybridization with the polynucleotide probe of the nucleotide array. According to this method, biomarkers are detected as an increased hybridization signal intensity, as compared with genes that are not affected upon administration of the therapeutic agent. Alternatively, biomarkers are identified by determining which polynucleotide probes, attached to the surface of the nucleotide array, form hybridized commplexes with the test sample from a primate administered a therapeutic agent but do not form hybridized complexes with the control sample from a primate that was not administered a therapeutic agent. Also, biomarkers may be identified by determining which polynucleotide probes, attached to the surface of the nucleotide array, form hybridized commplexes with the test sample from a primate administered a therapeutic agent but do not form hybridized complexes with the control sample from a primate that does not respond to a therapeutic agent.

Moreover, as indicated above, the nucleotide array of this invention may be used to determine a more target-specific therapeutic agent from an initial therapeutic agent. In this case, the targets of the initial therapeutic agent may be identified as above. The structure of the initial therapeutic agent may then be modified. Then, the modified therapeutic agent may be administered to the primate. The targets of the modified therapeutic agent may then be identified as above and then compared with the targets of the initial therapeutic agent.

Furthermore, as indicated above, the nucleotide array of this invention may be used to determine single nucleotide polymorphisms. In this case, the hybridization signal of the duplex between the polynucleotide probe and the test sample from a primate with an SNP will be reduced, as observed in the mismatch controls. Thus, the hybridization signal intensity will be reduced as compared with hybridization of the polynucleotide probe for the control sample that lacks the single nucleotide polymorphism.

A non-limiting example of systems in which the nucleotide array of the present invention may be used to assay the changes in gene expression levels resulting from the administration of a therapeutic agent, especially to determine the toxicity of the therapeutic agent, is a Cynomolgus monkey system to study human diseases. Especially relevant disease models utilizing a Cynomolgus monkey system are viral infections and vaccine efficacy, such as HIV, SARS, smallpox (variola), human influenza, tuberculosis, hepatitis, and Venezuelan equine encepyhalitis. Other relevant disease models utilizing a Cynomolgus monkey system are directed to studying coronary atherosclerosis, Parkinson's disease, osteoarthritis, Alzheimer's disease, and acute experimental autoimmune encephalomyelitis.

Another aspect of the present invention, as indicated above, is a method for normalizing data comprising (a) determining the signal intensity of the hybridized complex between the test or control sample and a polynucleotide probe that is complementary to, or a fragment of, a Cynomolgus monkey gene that is known not to be up- or down-regulated upon administration of a specific therapeutic agent; (b) averaging the signal intensity of the specific hybridized complex on different nucleotide arrays; (c) determining the ratio between the average signal intensity of the specific hybridized complex on all of the nucleotide arrays and the signal intensity of the specific hybridized complex on the nucleotide array of interest; and (d) adjusting the signal intensities of the hybridized complexes between the other hybridized complexes on the nucleotide array based upon the calculated ratio. In one embodiment of the present invention, the polynucleotide probe that is complementary to, or a fragment of, a Cynomolgus monkey gene that is known not to be up- or down-regulated upon administration of a specific therapeutic agent is complementary to, or a fragment of, any portion of any of SEQ ID NOS. 1-8881 or SEQ ID NOS. 9187-18598. In another embodiment of the present invention, the polynucleotide probe that is complementary to, or a fragment of, a Cynomolgus monkey gene that is known not to be up- or down-regulated upon administration of a specific therapeutic agent is any of SEQ ID NOS. 8882-9186.

In one embodiment of the present invention, the method of normalizing data additionally comprises (a) determining the signal intensity of the hybridized complex between the test or control sample and a polynucleotide probe that is complementary to, or a fragment of, a Rhesus monkey gene that is known not to be up- or down-regulated upon administration of a specific therapeutic agent; (b) averaging the signal intensity of the specific hybridized complex between test or control sample and the polynucleotide probe that is complementary to, or a fragment of, the Rhesus monkey gene, on different nucleotide arrays; (c) determining the ratio between the average signal intensity of the specific hybridized complex on all of the nucleotide arrays and the signal intensity of the specific hybridized complex on the nucleotide array of interest; and (d) adjusting the signal intensities of the hybridized complexes between the other hybridized complexes on the nucleotide array based upon the calculated ratio. In one embodiment of the present invention, the polynucleotide probe that is complementary to, or a fragment of, a Rhesus monkey gene that is known not to be up- or down-regulated upon administration of a specific therapeutic agent is complementary to, or a fragment of, any portion of any of SEQ ID NOS. 18599-35840 or SEQ ID NOS. 36075-43225. In another embodiment of the present invention, the polynucleotide probe that is complementary to, or a fragment of, a Rhesus monkey gene that is known not to be up- or down-regulated upon administration of a specific therapeutic agent is any of SEQ ID NOS. 35841-36074.

In yet another embodiment of the present invention, the method of normalizing data additionally comprises (a) determining the signal intensity of the hybridized complex between the test or control sample and a polynucleotide probe that is complementary to, or a fragment of, a human gene that is known not to be up- or down-regulated upon administration of a specific therapeutic agent; (b) averaging the signal intensity of the specific hybridized complex between test or control sample and the polynucleotide probe that is complementary to, or a fragment of, the human gene, on different nucleotide arrays; (c) determining the ratio between the average signal intensity of the specific hybridized complex on all of the nucleotide arrays and the signal intensity of the specific hybridized complex on the nucleotide array of interest; and (d) adjusting the signal intensities of the hybridized complexes between the other hybridized complexes on the nucleotide array based upon the calculated ratio. In one embodiment of the present invention, the polynucleotide probe that is complementary to, or a fragment of, a human gene that is known not to be up- or down-regulated upon administration of a specific therapeutic agent is complementary to, or a fragment of, any portion of any of SEQ ID NOS. 43450-48714. In another embodiment of the invention, the polynucleotide probe that is complementary to, or a fragment of, a human gene that is known not to be up- or down-regulated upon administration of a specific therapeutic agent is any of SEQ ID NOS. 43226-43449.

Furthermore, the nucleotide array of the present invention may be utilized in an in vitro system. The gene expression upon exposure of a therapeutic agent to an isolated cell line may be assayed by exposing the therapeutic agent to a cell line isolated from a primate, e.g., a cell line isolated from a human, a cell line isolated from a Cynomolgus monkey, or a cell line isolated from a Rhesus monkey, especially a cell line isolated from a non-human primate such as a Cynomolgus monkey or a Rhesus monkey, isolating the RNA from the cell line to yield a test sample, and hybridizing the test sample with the nucleotide array of interest. In one embodiment, the changes in gene expression are detected by comparing the hybridization pattern of the nucleotide array exposed to the test sample of a cell line from a primate exposed to a therapeutic agent with the hybridization pattern of a nucleotide array exposed to the control sample of a cell line from a primate that was not exposed to a therapeutic agent.

Examples of in vitro systems in which the nucleotide array of the present invention may be utilized are those directed to cancer, skin permeation, cytotoxicity, embryonic stem cell lines, expression of cytochrome P-450 and other metabolizing enzymes, endocrine disruption, genetic toxicity, metabolism-mediated toxicity, and hepatocyte models of liver toxicity.

The following examples are illustrative, but not limiting, of the methods of the present invention. Other suitable modifications and adaptations of the variety of conditions and parameters normally encountered in the field, and which are obvious to those skilled in the art, are within the spirit and scope of the invention.

All patents and publications cited herein are fully incorporated by reference herein in their entirety.

### EXAMPLES

### Example 1

### Generation of Cynomolgus EST Sequences

Libraries were generated from mRNA isolated from ten Macaca fascicularis tissues. The origin of the Macaca fascicularis was Indonesia.

Nine non-normalized, oligo-dT primed directionally clone libraries were generated from bone marrow, spleen, skin, thymus, heart, lung, liver, kidney, and lymph node. A normalized, oligo-dT primed library was generated from brain tissue. To manage the redundancy, the gene discovery rate was monitored after sequencing every 2000 reads from every library. Libraries with higher estimated gene discovery rate were sequenced at a greater depth compared to other libraries.

The specific tissue of the Macaca fascicularis was ground using a mortar and pestle in liquid nitrogen to form a fine powder. The finely ground powder was dissolved in TriZOL reagent and then homogenized with a Polytron Homogenizer. Following centrifugation, total RNA was precipitated from the supernatant using isopropyl alcohol. The RNA was resuspended in RNAse free water, quantitated using UV spectrophotometry, and analyzed by formaldehyde gel.

The mRNA was isolated from each total RNA sample by binding the mRNA to oligo-dT beads in a hybridization buffer, washing contaminating particles away with a wash buffer, and eluting the mRNA in RNAse free water.

The primary libraries were constructed by first strand cDNA synthesis of mRNA using an oligo-dT primer containing a rare restriction enzyme site and an RNAse H+ reverse transcriptase under proprietary conditions (Agencourt Bioscience Corporation).

Second strand cDNA was synthesized using standard methods of Gubler and Hoffman. See U. Gubler & B. J. Hoffman, Gene, 25:263-69 (1983), herein incorporated by reference.

The cDNA was analyzed by agarose gel. The size of the cDNA was selected to be greater than 1.2 kb. The selected cDNA was purified and then ligated into a suitably digested pAgen vector. The ligations were transformed into T1 phage resistant DH10B cells.

The transformed cells were then plated to determine titer. Twenty clones were selected for digestion to determine average insert size.

Normalized cDNA libraries were prepared by in vitro transcription of primary library DNA to make biotinylated RNA anti-sense to the cloned cDNA. In addition, phagemid infection of the primary library was used to produce single stranded DNA circles containing the sense strand of the cDNA clone. The antisense RNA and sense DNA were hybridized using conditions that favor the hybridization of abundantly expressed sequences. Double stranded structures were removed via strepavidin/phenol extraction leaving single stranded DNA circles representing the normalized library. The circles are repaired to double strand form and electroporated back into TI phage resistant DH10B E. coli cells.

A total of 100,000 3′ ESTs were generated and subjected to quality control using a combination of the software LUCY and in-house perl scripts.

Specifically, the libraries were plated to a density of 1000-2000 colonies/plate and then picked using an automated picking robot into bar-coded 384-well glycerol plates. The 384-well glycerol plates were processed through an automated DNA preparation pipeline that utilized the SprintPrep™ technology.

Sequencing was then accomplished by standard sequencing methods utilizing BigDye™ chemistry on ABI 3730 sequencing machines. The sequences results were downloaded from the machines and processed through the PHRED basecalling program. PHRED is a software that reads the bases and assigns a quality value for each base in the trace. See B. Ewing & P. Green, Genome Research, 8:186-94 (1998).

The average length of the raw sequence was 824 bp. The average PHRED score/trace was 32.

The quality control steps included removal of low quality, vector, contaminant, ribosomal, and mitochondrial traces, and clipping of traces with low quality ends and/or presence of vector sequences.

The vector sequences and low quality bases were removed using LUCY. See H. H. Chou & M. H. Holmes, Bioinformatics, 17:1093-1104 (2001).

The default settings were used for both PHRED and LUCY.

The sequences were next screened for E. coli, rRNA, mitochondrial DNA using Cross-match (minmatch=14; miniscore=100; screen=0). Cross-match is an implementation of the Smith-Waterman-Gotoh algorithm that may be used to compare nucleic acid sequences.

After the quality control steps, a total of 80,147 good quality sequences were generated. These good quality sequences were used for further analysis. The average length of the good quality sequences was 620 bp. The average PHRED score/trace of the good quality sequences was 43. The Cynomolgus sequences mapped to about 9,000 human genes.

The heart library did exhibit a high content of mitochondrial genes.

### Example 2

### Clustering of Cynomolgus EST Sequences

The good quality ESTs were clustered and assembled using the combination of CAT (version 4.5) and PHRAP softwares. See Burke et al., Genome Research 8:276-90 (1998). The default settings were used except for the following: (a) minimum overlap (d2_window) was set to 75 bp, (b) overlap identity (d2_string) was set to 93%, (c) the filter option was turned on (low_complexity=1; simple=1, repeats=1). Repeats were screened against repeat database present in the CAT software, as well as against the Repbase database. See J. Jurka, Current Opinion in Structural Biology, 8:333-37 (1998).

Two sequences were brought into the same cluster if they had an overlap of 75 bp and 93% identity. Unmatched overhangs in the cluster were split by PHRAP, which was used to create the consensus sequences. A majority of the singletons identified in the penultimate round of clustering were resequenced and added to the final assembly.

The assembled sequences were then checked for orientation. The unigenes were compared to Ensembl human cDNAs using BLASTN with an Evalue<10⁻⁵. The unigenes were also compared against the human genome using BLAT. The default settings were used for BLAT.

The unigenes were reverse complemented if the orientation was inconsistent with either the orientation of the Ensembl human cDNA or the orientation of the annotated genes in the genome. In those cases, both the forward and reverse sequences were used in the design of the chip.

A total of 80,147 ESTs were assembled into 16,357 unigenes. Of these, 3,335 remained as singletons.

The assembly was examined to understand the distribution of ESTs among the unigenes. A total of 11,702 unigenes (71.5%) contained more than one, but less than 10, EST members. A total of 109 unigenes had greater than 50 EST members.

ESTs from the brain tissue were represented in 41% of the unigenes. At the lower end, the ESTs from the heart tissue were represented in 9.5% of the unigenes and the ESTs from the liver tissue were represented in 10.9% of the unigenes. The ESTs from the other tissues were more or less evenly represented.

The percent of library-specified unigenes were computed to investigate the specificity of transcripts across the sampled tissues. A library-specific unigene was defined as a unigene with EST members unique to one of the sequenced libraries. A total of 9,588 unigenes (58.6%) contained members unique to a library. 3,032 (18.5%) unigenes were specific to brain tissue. The specificity for other libraries ranged from 2% (heart) to 6.3% (lymph node).

The rate of gene discovery was evaluated for each library by calculating the percent unigenes (total number of contigs and singletons divided by the number of good quality ESTs). The normalized brain library had the highest rate of gene discovery and was deeply sampled. Seven of the remaining nine non-normalized libraries exhibited similar levels of redundancy within the libraries. The liver and heart libraries exhibited higher levels of redundancy and were not deeply sampled.

After several rounds of sequencing, the percent unigenes across libraries ranged from 35% to 52%, with most of the libraries clocking at the higher end of this range.

### Example 3

### Identification of Cynomolgus Gene Sequences

Publicly available Macaca fascicularis sequences were also utilized in the construction of the monkey chip. A total of 2,773 Macaca fascicularis transcript sequences were obtained from Genbank. The sequences were processed as described above, except that the sequences were not processed using PHRED. These sequences were assembled separately using the methods described above. The assembly process resulted in 2,170 sequences.

### Example 4

### Generation of Rhesus EST Sequences

Macaca mullata mRNA sequences available in Genbank were utilized in the construction of the monkey chip. A total of 20,139 Macaca mullata transcript sequences were obtained from GenBank. However, trace quality information was not available for most of these sequences. Also, most of the Macaca mullata transcript sequences in Genbank appeared to be complementary to the 5′ end.

These mRNA sequences were processed as described in Example 1, with the exception that the sequences were not processed using PHRED.

### Example 5

### Identification of Rhesus Gene Sequences

The sequences and accompanying files for 2,330,205 whole genome shotgun sequences from the Human Genome Sequencing Center at Baylor College of Medicine were downloaded from the NCBI traces archive. The 5′ and 3′ ends of the sequences were trimmed to remove any vector or poor quality sequence. The BLAT results were post-processed, as described below, to omit potential intron regions from the sequence.

Each BLAT alignment can have multiple blocks (analogous to HSPs in BLAST) associated with it. For each BLAT hit, the total score was divided by the sum of all block lengths to calculate the score per nucleotide. If adjacent blocks were separated by a gap that represented less than 5% of the sum of their lengths, those blocks were merged into a single block. Consequently, multi-blocked entries were consolidated into one or more single block entries. Then, the scores of the single block entries were calculated as the total block length multiplied by the entry's score per nucleotide. From these results, the highest-scoring human genome block was identified for each Rhesus genomic sequence (RGS).

The genomic locus of each top scoring hit was cross-referenced against the locations of known human genes to determine whether or not the RGS likely represented a coding sequence (“RGS-Coding”) or an UTR sequence (“RGS-putative 3′UTR”). An RGS that did not align with a locus at which an exon or UTR was annotated were removed from further analysis. The remaining RGS's were BLASTed against all Ensembl build #33 cDNAs. The BLAST and BLAT results were compared to each other to ensure that the gene that was found using the BLAT approach was also present in the BLAST results.

Each RGS was then consolidated into transcripts using human transcripts as a template. The segment of each RGS that aligned to an Ensembl transcript was cut out (e.g., alignment with the transcript may be interrupted by an intron). If more than one RGS covered the same locus of a gene, the sequences were consolidated using PHRAP. Then, for the final files of assembled RGS's, if the Rhesus DNA segments did not exhibit a continuous alignment across a human gene, the sequence segments were separated with the appropriate number of Ns.

For the more than half of the human Ensembl genes that do not have defined 3′ UTRs, the annotated 3′ coordinates correspond to the stop codon of the gene. Because the DNA chip technology depends on the proximity to the 3′ end of the transcript, the longest RGS that aligned to a sequence within 1 kilobase downstream of each stop codon of the gene was also collected. These sequences were considered to be putative 3′ UTRs.

The 3 sets of Rhesus sequences needed to be merged (the Rhesus public ESTs described in Example 3 and the RGS-putative 3′ UTRs and RGS-Coding described in Example 4) to avoid redundant, overlapping sequences. The merger approach involved having the EST sequences take precedence over either the RGS-putative 3′ UTRs or the RGS-Codings, because the EST sequences were known, expressed sequences.

RGS-Codings were BLASTed against a database of public Rhesus EST sequences. If sequences that represent the same Ensembl transcript were found to overlap by more than 50 bp, with a greater than 90% identity, the RGS-Coding was removed from the master sequences file. The Rhesus ESTs were pooled with the remaining RGS-Codings and these sequences were BLASTed against a database of RGS-putative 3′ UTRs (10⁻⁵). Any RGS-putative 3′ UTR that was hit was removed from the master file of the RGS-putative 3′ UTR sequence file.

A total of 18,897 sequences were used for further analysis. These sequences were clustered and assembled as described in Example 1, resulting in 10,875 unigenes, of which 7,317 were singletons. There were 6,886 ENSEMBL transcripts were covered by the Rhesus unigenes, some of them were covered by more than one unigene. Additionally, 2,161 Rhesus unigenes did not map to any ENSEMBL transcripts.

The summary of the transcription data is shown in Table 1 below.

TABLE 1

| | | Tissues | | | | | | | | | | | |
|---|---|---|---|---|---|---|---|---|---|---|---|---|---|
| | | | | Cyno | | Cyno | | Cyno | | Cyno | | Cyno | |
| | Total | All | | heart | | brain | | lung | | liver | | kidney | |
| Sequences | Num Seq | Number | Percent | Number | Percent | Number | Percent | Number | Percent | Number | Percent | Number | Percent |
| All | 51724 | 48365 | 94% | 29252 | 57% | 31534 | 61% | 28379 | 55% | 27796 | 54% | 24274 | 47% |
| Human | 5606 | 4799 | 86% | 2289 | 41% | 2487 | 44% | 2013 | 36% | 1947 | 35% | 1680 | 30% |
| Cyno | 20426 | 19925 | 98% | 13882 | 68% | 14962 | 73% | 14001 | 69% | 13758 | 67% | 12074 | 59% |
| Rhesus | 27074 | 25006 | 92% | 14211 | 52% | 15229 | 56% | 13528 | 50% | 13226 | 49% | 11588 | 43% |

| | | Rhesus | | Rhesus | | Rhesus | | Rhesus | | Rhesus | |
|---|---|---|---|---|---|---|---|---|---|---|---|
| | Total | heart | | brain | | lung | | liver | | kidney | |
| Sequences | Num Seq | Number | Percent | Number | Percent | Number | Percent | Number | Percent | Number | Percent |
| All | 51724 | 30451 | 59% | 31991 | 62% | 30674 | 59% | 24705 | 48% | 28000 | 54% |
| Human | 5606 | 2385 | 43% | 2517 | 45% | 2239 | 40% | 1722 | 31% | 1984 | 35% |
| Cyno | 20426 | 14086 | 69% | 14974 | 73% | 14688 | 72% | 12083 | 59% | 13649 | 67% |
| Rhesus | 27074 | 14974 | 55% | 15533 | 57% | 14774 | 55% | 11752 | 43% | 13312 | 49% |

### Example 6

### Polynucleotide Probe Design and Selection

Polynucleotide probes were designed using standard design guidelines and algorithms as outlined in Affymetrix literature “GeneChip® Custom Array Design Guide” and “New Statistical Algorithms for Monitoring Gene Expression on GeneChip® Probe Arrays”, herein incorporated by reference. The polynucleotide probes were designed with a 3′ bias in which the terminal 600 bases of the transcript were targeted.

Each polynucleotide probe set was assigned a quality score based on predicted hybridization efficiency. The quality score was lowered if the polynucleotide probe was likely to cross-hybridize or overlapped with another polynucleotide probe.

In addition to the quality score, whether a particular polynucleotide probe was included on the nucleotide array was dependent upon the type of probe set—whether the probe set was unique, identical, or mixed. A unique probe set is a probe set in which each polynucleotide probe hybridizes to only one nucleic acid. An identical probe set is a probe set in which the polynucleotide probes will hybridize to more than one nucleic acid. A mixed probe set is a probe set in which different polynucleotide probes within the probe set hybridize with differing nucleic acid sequences.

All polynucleotide probe sets with a quality score ≧2.34 were included on the nucleotide array. Then, the highest scoring unique and identical probe sets containing polynucleotide probes that were complementary to, or were fragments of, Cynomolgus monkey genes or Rhesus monkey genes, but had a quality score lower than 2.34 were included on the nucleotide array until there was no additional space on the nucleotide array. The final cut-off score was 1.7437.

Multiple polynucleotide probe sets exist on the nucleotide array for some nucleic acids due to redundancy between the Cynomolgus monkey and Rhesus monkey sequences.

To maximize the nucleotide array content, human sequences for which no primate ortholog was identified were included on the nucleotide array.

### Example 7

### Nucleotide Array

The nucleotide array may be prepared by any method known to a person of ordinary skill in the art. Particular examples of such methods that may be utilized for each of these steps are disclosed in Affymetrix literature “Array Design for the Gene Chip® Human Genome U133 Set”, U.S. Patent Application No. 2004/0259124, and U.S. Pat. Nos. 5,412,084; 6,147,205; 6,262,216, 6,310,189; 5,889,165; and 5,595,098, incorporated herein by reference.

Specifically, the nucleotide array was prepared utilizing photographic methods. See J. W. Jacobs & S. P. Fodor, Trends in Biotechnology, 12:19-26 (1994); L. T. Mazzola & S. P. Fodor, Biophys. J, 68:1653-1660 (1995), herein incorporated by reference.

The nucleotide array contained 51,724 total probesets. Specifically, 19,917 probesets contained polynucleotide probes that were complementary to, or fragments of, Cynomolgus monkey sequences. The nucleotide array contained 12,178 probesets that contained polynucleotide probes that were complementary to, or fragments of, Rhesus monkey genomic sequences (“RGS-Coding”). The nucleotide array also contained 9,111 and 4,983 probesets containing polynucleotide probes that were complementary to, or fragments of, Rhesus ESTs and Rhesus monkey putative 3′UTR (“RGS-putative 3′UTR”), respectively. Finally, the nucleotide array contained 5,526 probesets containing polynucleotide probes that were complementary to, or fragments of, human genomic sequences, as well as 9 negative controls.

Each probeset consisted of 13 polynucleotide probes per sequence. Each polynucleotide probe was 25 nucleotides in length.

The nucleotide array was designed such that 40,174 probesets were unique sets that only recognized one nucleic acid, 7,421 probesets were identical sets that would recognize more than one nucleic acid, and 2,209 probesets were mixed sets in which some polynucleotide probes would cross-hybridize with other nucleic acid sequences.

Of the 1,739 human Tox genes, 1,689 were attached to the surface of the nucleotide array.

Several control polynucleotide probe sets were also included on the nucleotide array to assist in evaluating performance of the nucleotide array and to scale the hybridization intensity data.

First, non-eukaryotic hybridization controls (bioB, bioC, bioD, and cre) were included on the nucleotide array as normalization controls to monitor the performance of the hybridization, staining, and washing procedures.

Second, polynucleotide probe sets for several B. subtilis genes (dap, lys, phe, and thr) were included on the nucleotide array to assess the labeling process independent of the sample RNA quality.

Third, edge controls, corner checkerboards, and a center cross were included on each nucleotide array for grid alignment purposes.

Fourth, polynucleotide probe sets representing beta-actin and glyceraldehyde-3-phosphate dehydrogenase were also included on the nucleotide array. Polynucleotide probe sets were designed to the 5′, middle, and 3′ regions of these transcripts to control for RNA quality and labeling efficiency.

Polynucleotide probe sets representing 100 normalization control genes that are homologous to those included on the human U133 Plus 2.0 array were included on the nucleotide array of the present invention for cross-array comparisons.

### Example 8

### Sample Preparation for Gene Expression Analysis

The nucleotide array was used to examine the basal gene expression in the cerebellum, heart, liver, kidney, and lung tissues from naïve female Cynomolgus monkeys and male Rhesus monkeys.

The particular tissue of interest was homogenized in freshly prepared RLT buffer (Qiagen). The total RNA from the tissue was isolated using RNeasy columns (Qiagen) according to the manufacturer's protocol.

The isolated RNA was reverse transcribed into double-stranded cDNA. The double-stranded cDNA was reverse transcribed using a T7-oligo(dT) primer, SuperScript™ II reverse transcriptase, and the Affymetrix One Cycle cDNA Synthesis Kit. The steps of the reverse transcription reaction were performed as outlined in the Affymetrix One Cycle cDNA Synthesis Kit. The synthesized cDNA was purified using spin columns from the Affymetrix Sample Cleanup Module, according to the manufacturer's instructions.

Biotinylated cRNA was synthesized from the double-stranded cDNA utilizing the Affymetrix IVT Labeling Kit. The purified cDNA was primed using a T7 primer and T7 RNA polymerase. All of the in vitro transcription reactions were performed according to the manufacturer's protocol. The transcribed, biotinylated cRNA was then purified using spin columns.

The biotinylated cRNA was then fragmented using the Affymetrix Sample Cleanup Module in 5× Fragmentation Buffer, in accordance with the manufacturer's instructions.

Then, the fragmented biotinylated cRNA was hybridized overnight with the polynucleotide probes of the nucleotide array, as detailed in the Affymetrix GeneChip® Expression Analysis Technical Manual 701025/Rev 6 (Affymetrix, Santa Clara, Calif.).

The nucleotide arrays containing the hybridized complexes were washed and stained using the EukGE-WS2 antibody amplification protocol as detailed in the Affymetrix GeneChip® Expression Analysis Technical Manual 701025/Rev 6 (Affymetrix, Santa Clara, Calif.).

The samples were processed according to the general procedures outlined within the GeneChip® Operating Software (“GCOS”) and Rosetta Resolver v4.0.1.78 software packages. The library files for the nucleotide array were uploaded into Resolver for each of the Cynomolgus and Rhesus monkeys. One specific Cynomolgus monkey and one specific Rhesus monkey pattern file was created.

Data from each scan were uploaded into Resolver manually using the GeneChip® Migration Wizard. The appropriate pattern specified was dependent upon the specifies from the fragmented, biotinylated RNA hybridized to the nucleotide array was derived.

The software's error model was applied as the default “intensity profiles” were built, as described in Rosetta Technical Note (2001). Rosetta Resolver Application Error Model for Affymetrix GeneChip Microarray Data. (Rosetta Biosoftware, Seattle, Wash.). The intensity profiles for each nucleotide array hybridized with Cynomolgus fragmented, biotinylated cRNA were selected and a trend analysis was performed. Each profile was labeled with an integer from 0 to 21, with the profiles sorted in alphabetical order. Once the trend was built, the calculated p-values for each transcript were exported as a tab separated text file with an .xls extension using the data export tool. These same steps were utilized in building a trend for the Rhesus scans.

The total fluorescence intensities of each nucleotide array were scaled to 250 prior to comparison analysis. The data were filtered in DMT for signal log ratio (−1_SLR_{—}1) and gene detection (designated as “present” or “absent”). In this experiment, all transcripts called “marginal” by the Affymetrix algorithm were considered “present”.

The summary of the transcription data is shown in Table 1 above in Example 5.

The presence or absence of each transcript in the tissue of interest is shown in Table 2.

### Example 9

### Toxicogenomic Screening Using the Nucleotide Array

A therapeutic agent will be administered to a non-human primate, e.g., a Cynomolgus or Rhesus monkey, according to an appropriate treatment protocol and dosing schedule. A separate group of non-human primates of the same species will be designated as the control group. The members of the control group will not be administered the therapeutic agent of interest.

At the end of the treatment protocol, the non-human primates of both the test and the control groups will be sacrificed. The following tissues may be collected as the biological sample(s) for toxicogenomic and histopathology analysis from the non-human primates of both the test and control groups: liver, pancreas, kidneys, uterus, testes, thyroid, brain, heart, ovaries, spleen, thymus, prostate, colon, and/or lungs. Each sample of interest for the toxicogenomics analysis will be submerged in RNALater TissueProtect Tubes (Qiagen) and cut into small pieces. Each sample of interest will remain in the solution for at least 30 minutes at room temperature.

The RNA from the biological sample from the test and control non-human primates will be isolated. The RNA will be reverse transcribed to generate cDNA. The generated cDNA will be transcribed in vitro to generate labeled RNA. The labeled RNA will be fragmented to generate the test and control samples. The labeled RNA of the test and control samples will be hybridized to the nucleotide array containing the polynucleotide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449, immobilized to the solid surface of the nucleotide array. The nucleotide array will then be washed and stained to visualize the hybridization pattern. The hybridization pattern of the test group will then be compared with the hybridization pattern of the control group to determine which non-human primate genes were up- or down-regulated in response to the administered therapeutic agent.

The RNA isolation, reverse transcription steps, in vitro transcription, RNA labeling, fragmentation, hybridization, washing, and staining steps may be any method known by a person of ordinary skill in the art. Particular examples of such methods that may be utilized for each of these steps are disclosed in U.S. Pat. Nos. 5,837,832; 6,306,643 B1; 6,309,823 B1, 6,344,316 B1; and 6,410,229 B1, incorporated herein by reference.

### Example 10

### Use of the Nucleotide Probe in a Primate System

A non-human primate, e.g., a Cynomolgus monkey or a Rhesus monkey, will be infected with a particular virus of interest, including, but not limited to HIV, SIV, SARS, human influenza, small pox (variola), tuberculosis, hepatitis, or Venezuelan equine encaphalitis. Then, the non-human primate may be treated with a therapeutic agent according to an appropriate treatment protocol and dosing schedule. A separate group of non-human primates of the same species will be designated as the control group. The members of the control group will not be administered the therapeutic agent of interest.

At the end of the treatment protocol, the non-human primates of both the test and the control groups will be sacrificed and the biological sample(s) of the test and control non-human primates will be collected and prepared as described above in Example 9. The test and control samples will be prepared as described above in Example 9. Then, the labeled RNA of the test and control samples will be hybridized to the nucleotide array containing the polypeptide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449, as described above in Example 9. The hybridization pattern of the test group will then be compared with the hybridization pattern of the control group to determine which non-human primate genes correlate to a response to the administered therapeutic agent and/or toxic reactions to the therapeutic agent in a non-human primate infected by the virus of interest.

### Example 11

### Serum Analysis to Identify Genes Correlating to Drug Response or Toxicity Using the Nucleotide Array

A therapeutic agent will be administered to a non-human primate, e.g., a Cynomolgus or Rhesus monkey, according to an appropriate treatment protocol and dosing schedule. A separate group of non-human primates of the same species will be designated as the control group. The members of the control group will not be administered the therapeutic agent of interest.

At the beginning and end of the treatment period, serum samples will be collected from the non-human primates of both the test and control groups. The RNA will be isolated from the serum samples using QIAamp (Qiagen) according to the manufacturers' instructions. The test and control samples will be prepared as described above in Example 9. Then, the labeled RNA of the test and control samples will be hybridized to the nucleotide array containing the polypeptide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449, as described above in Example 9. The hybridization pattern of the test group will then be compared with the hybridization pattern of the control group to determine which non-human primate genes correlate to a response to the administered therapeutic agent and/or toxic reactions to the therapeutic agent.

### Example 12

### Use of the Nucleotide Probe in an In Vitro System

An in vitro cell culture of a non-human primate, e.g., a Cynomolgus monkey or a Rhesus monkey, will be exposed to a therapeutic agent according to an appropriate treatment protocol and dosing schedule. A separate in vitro cell culture of the same species will be designated as the control group. The cell culture of the control group will not be administered the therapeutic agent of interest.

At the end of the treatment protocol, the cells will be harvested using an appropriate protocol. The RNA will be isolated from the harvested cells and the test and control samples will be prepared as described above in Example 9. Then, the labeled RNA of the test and control samples will be hybridized to the nucleotide array containing the polypeptide probes complementary to, or fragments of, any portion of any of SEQ ID NOS. 1-8881, SEQ ID NOS. 9187-18598, SEQ ID NOS. 18599-35840, SEQ ID NOS. 36075-43225, and SEQ ID NOS. 43450-48714, as well as the polynucleotide probes of any of SEQ ID NOS. 8882-9186, SEQ ID NOS. 35841-36074, and SEQ ID NOS. 43226-43449, as described above in Example 9. The hybridization pattern of the test group will then be compared with the hybridization pattern of the control group to determine which non-human primate genes correlate to a response to the administered therapeutic agent and/or toxic reactions to the therapeutic agent in a non-human.

### Example 13

### Expression of Tissue Specific Genes

Samples of right ventricle and cerebellum tissues were taken from a Cynomolgus and a Rhesus monkey. These samples were hybridized to the nucleotide array of Example 7. A selection of genes that were expressed in one tissue and not the other, for both species, was clearly related to the organ in question.

Study Design

Right ventricle and cerebellum tissues were selected for transcript profiling, based on a large difference in transcripts present in one tissue and not the other. These organs were obtained from a Rhesus macaque and a Cynomolgus macaque and samples were removed for transcript profiling. Each tissue from each species was hybridized to six nucleotide arrays from Example 7, for a total of 24 nucleotide arrays used for the experiment.

Processing of Samples and Quality Control for Transcript Profiling

Sample homogenization, total RNA extraction, labeling, and hybridization procedures were performed by GeneLogic according to Affymetrix recommended protocols and standard procedures. Quality control consisted of measurement of A₂₆₀/A₂₈₀ ratios to assess protein contamination. RNA degradation and genomic DNA contamination were also assessed, using the Agilent Bioanalyzer.

cDNA and labeled cRNA were prepared using Affymetrix kits according to standard procedures. cRNA A₂₆₀/A₂₈₀ ratios were measured to check quality. The cRNA samples were then fragmented, hybridized onto the nucleotide arrays of Example 7 and scanned. Scan quality was assessed by examining background intensity, scaling factor, percent present calls, GAPDH 3′/5′ ratios, and beta actin 3′/5′ ratios. Values that were outside 3 standard deviations from the mean were flagged.

(a) Data Acquisition and Reduction

Affymetrix GeneChip Operating Software (“GCOS”) was used for instrument control and data acquisition according to the manufacturer's instructions. Affymetrix Technical Note (2003). Standardized Assays and Reagents for GeneChip Expression Analysis. Part Number 701192/Rev3 (Affymetrix, Santa Clara, Calif.). Briefly, after scanning the microarray, GCOS identified and utilized the signal from spiked-in controls (Oligo B2) to align a grid to the scanned image. Intensity values for each feature were calculated by taking the median intensity of all pixels assigned to the feature. Global normalization was performed to compensate for chip-to-chip variability by scaling the intensity values on each chip to a median of 250. These scaled data were saved in a CEL file, which includes the X and Y coordinates of each probe feature, intensity mean and standard deviation, the number of pixels used for calculating intensity values, and background intensity. The data in the .CEL file and the associated images were subsequently manually uploaded into Rosetta Resolver using the Rosetta GeneChip Migration Wizard.

“Experiment Definitions” (“ED's”) were constructed in Rosetta Resolver to facilitate data processing and analysis. All definitions used the Affymetrix—Intensity Profile Builder visual script to compute error model statistics and p-values, the Affymetrix—Intensity Experiment Builder (no Reporters) visual script to perform normalization and combine intensities across chips, and the Affymetrix—Default Ratio Builder visual script to create ratios and compute p-values. Rosetta Technical Note (2001). Rosetta Resolver Application Error Model for Affymetrix GeneChip Microarray Data. (Rosetta Biosoftware, Seattle, Wash.). The majority of analysis was performed using the individual ratios or intensities; however, the average fold change or intensity results from the group analyses are reported for clarity.

(b) Data Analysis

The list of probe sets corresponding to transcripts that were uniquely expressed in each tissue, for each species, was generated. The Rosetta Resolver intensity p-value (Rosetta Technical Note (2001). Rosetta Resolver Application Error Model for Affymetrix GeneChip Microarray Data. (Rosetta Biosoftware, Seattle, Wash.)) was used to determine whether the transcripts were expressed or not. A transcript with a p-value ≦0.01 was considered “present”; a transcript with a p-value >0.04 was considered “absent”. For each species, the list of probe sets that was present in all six replicates of one tissue, and absent in all six replicates of the other tissue was selected.

The annotations of these probe sets, derived from BLAST searches across the human and other genomes, was obtained. These lists were compared with the list of genes present in human cerebellum and not ventricle, or present in ventricle and not cerebellum, obtained from the ASCENTA database (https://ascenta.genelogic.com). Genes intersecting between all three species (separately for each tissue) were found. Those specifically related to either the brain or heart are shown in the results section below.

Results

Genes uniquely expressed in both Rhesus and Cynomolgus monkeys, in each tissue type (cerebellum or ventricle) specifically related to the organ in question, are shown in Tables 3 and 4 below.

TABLE 3
Cerebellum Specific Genes Consistent across Human,
Cynomolgus Monkey, and Rhesus Monkey

| SEQ ID NO | SEQ ID NO | | |
|---|---|---|---|
| (Rhesus | (Cynomolgus | | |
| Monkey) | Monkey) | Gene Name | Description |
| 16762 | 16762 | CTNND2 | catenin (cadherin-associated protein), delta 2 (neural |
| | | | plakophilin-related arm-repeat protein) |
| 19111 | 19111 | FABP7 | fatty acid binding protein 7, brain |
| 26023 | 26023 | GAD1 | glutamate decarboxylase 1 (brain, 67 kDa) |
| | 31000 | | |
| 27765 | 27765 | MT3 | metallothionein 3 (growth inhibitory factor (neurotrophic)) |
| 13908 | 13908 | NEF3 | neurofilament 3 (150 kDa medium) |
| 35417 | 15856 | NEFH | neurofilament, heavy polypeptide 200 kDa |
| 15856 | | | |
| 13197 | 13197 | NEFL | neurofilament, light polypeptide 68 kDa |
| 9477 | 9477 | | |
| 30232 | 30232 | NEUROD1 | neurogenic differentiation 1 |
| 9742 | 9742 | NPTX1 | neuronal pentraxin I |
| 27576 | 27576 | | |
| 28290 | 28290 | NRXN2 | neurexin 2 |
| 21336 | 21336 | NRXN3 | neurexin 3 |
| 27842 | 27842 | | |
| 45104 | 45104 | | |
| 17996 | 20511 | NTRK2 | neurotrophic tyrosine kinase, receptor, type 2 |
| 21471 | 21471 | RIMS2 | regulating synaptic membrane exocytosis 2 |
| 34809 | 34809 | SGNE1 | secretory granule, neuroendocrine protein 1 (7B2 protein) |
| 14873 | 14873 | | |
| 24164 | 32081 | SNAP25 | synaptosomal-associated protein, 25 kDa |
| 32081 | 11319 | | |
| 11319 | | | |
| 13915 | 13915 | SV2A | synaptic vesicle glycoprotein 2A |
| 22387 | 22387 | | |
| 31843 | 31843 | SV2B | synaptic vesicle glycoprotein 2B |
| 22443 | 22443 | SYN2 | synapsin II |
| 29443 | 29443 | | |
| 27656 | 27656 | SYNPR | synaptoporin |
| 9722 | 9722 | | |
| 12578 | 12578 | SYT1 | synaptotagmin I |
| 16039 | 16039 | SYT4 | synaptotagmin IV |
| 25031 | 25031 | | |

TABLE 4
Ventricle Specific Genes Consistent across Human,
Cynomolgus Monkey, and Rhesus Monkey

| SEQ ID NO | SEQ ID NO | | |
|---|---|---|---|
| (Rhesus | (Cynomolgus | | |
| Monkey) | Monkey) | Gene Name | Description |
| 12401 | 12401 | CMYA4 | cardiomyopathy associated 4 |
| 34797 | | | |
| 34539 | 34539 | CMYA5 | cardiomyopathy associated 5 |
| 20496 | 20496 | FABP3 | fatty acid binding protein 3, muscle and heart (mammary- |
| | | | derived growth inhibitor) |
| 26129 | 26129 | JUP | junction plakoglobin |
| 12605 | 12605 | MYBPC3 | myosin binding protein C, cardiac |
| 27853 | 27853 | MYH6 | myosin, heavy polypeptide 6, cardiac muscle, alpha |
| | | | (cardiomyopathy, hypertrophic 1) |
| 12302 | 26413 | MYH7 | myosin, heavy polypeptide 7, cardiac muscle, beta |
| 26413 | 12302 | | |
| 11571 | 11571 | MYL2 | myosin, light polypeptide 2, regulatory, cardiac, slow |
| 30554 | | | |
| 30229 | 30229 | MYL3 | myosin, light polypeptide 3, alkali; ventricular, skeletal, slow |
| 10572 | 32928 | MYOZ2 | myozenin 2 |
| 32928 | 10572 | | |
| 11930 | 34885 | NRAP | nebulin-related anchoring protein |
| 34885 | | | |
| 10712 | 10712 | PLN | phospholamban |
| 9320 | 9320 | | |
| 16190 | 16190 | POPDC2 | popeye domain containing 2 |
| 26858 | 26858 | RPL3L | ribosomal protein L3-like |
| 17661 | 17661 | SOD2 | superoxide dismutase 2, mitochondrial |
| 15825 | 15825 | TCAP | titin-cap (telethonin) |
| 11100 | 11100 | TNNT2 | troponin T2, cardiac |
| 9803 | 9803 | TPM1 | tropomyosin 1 (alpha) |
| 42601 | 42601 | TTN | titin |
| 34710 | 34710 | | |
| | 37715 | | |
| 12401 | 12401 | CMYA4 | cardiomyopathy associated 4 |
| 34797 | | | |
| 34539 | 34539 | CMYA5 | cardiomyopathy associated 5 |

The genes shown in Tables 3 and 4 above were expected to be expressed in the specified tissue. Therefore, the results shown in Tables 3 and 4 validate the nucleotide array of Example 7.

1. A nucleotide array for detecting changes in gene expression upon administration of a therapeutic agent comprising a plurality of polynucleotide probes complementary to, or fragments of, Cynomolgus monkey genes, wherein each polynucleotide probe is immobilized to a discrete and known spot on a solid support.

2. The nucleotide array of claim 1, wherein said polynucleotide probes are complementary to, or fragments of, any portion of an ortholog of a human gene.

3. The nucleotide array of claim 2, wherein said polynucleotide probes are complementary to, or fragments of, any portion of a homolog to a human Tox gene.

4. The nucleotide array of claim 1, wherein said polynucleotide probes are complementary to, or fragments of, any portion of any of SEQ ID NOS. 1-8881 or SEQ ID NOS. 9187-18598.

5-6. (canceled)

7. The nucleotide array of claim 1, wherein said polynucleotide probes are any of SEQ ID NOS. 8882-9186.

8. The nucleotide array of claim 1, wherein said nucleotide array additionally comprises at least one polynucleotide probe complementary to, or a fragment of, any portion of any human gene.

9-10. (canceled)

11. The nucleotide array of claim 2, wherein said nucleotide array additionally comprises at least one polynucleotide probe complementary to, or a fragment of, any portion of any human gene.

12-13. (canceled)

14. The nucleotide array of claim 3, wherein said nucleotide array additionally comprises at least one polynucleotide probe complementary to, or a fragment of, any portion of any human gene.

15-16. (canceled)

17. The nucleotide array of claim 4, wherein said nucleotide array additionally comprises at least one polynucleotide probe complementary to, or a fragment of, any portion of any human gene.

18. The nucleotide array of claim 17, wherein said polynucleotide probe from a human gene is complementary to, or a fragment of, any portion of any of SEQ ID NOS. 43450-48714.

19. The nucleotide array of claim 17, wherein said polynucleotide probe from a human gene is any of SEQ ID NOS 43226-48714.

20-25. (canceled)

26. The nucleotide array of claim 7, wherein said nucleotide array additionally comprises at least one polynucleotide probe complementary to, or a fragment of, any portion of any human gene.

27. The nucleotide array of claim 26, wherein said polynucleotide probe from a human gene is complementary to, or a fragment of, any portion of any of SEQ ID NOS. 43450-48714.

28. The nucleotide array of claim 26, wherein said polynucleotide probe from a human gene is any of SEQ ID NOS 43226-48714.

29. The nucleotide array of claim 1, wherein said nucleotide array additionally comprises at least one polynucleotide probe complementary to, or a fragment of, any portion of any Rhesus monkey gene.

30-32. (canceled)

33. The nucleotide array of claim 2, wherein said nucleotide array additionally comprises at least one polynucleotide probe complementary to, or a fragment of, any portion of any Rhesus monkey gene.

34-36. (canceled)

37. The nucleotide array of claim 3, wherein said nucleotide array additionally comprises at least one polynucleotide probe complementary to, or a fragment of, any portion of any Rhesus monkey gene.

38-40. (canceled)

41. The nucleotide array of claim 4, wherein said nucleotide array additionally comprises at least one polynucleotide probe complementary to, or a fragment of, any portion of any Rhesus monkey gene.

42. The nucleotide array of claim 41, wherein said polynucleotide probe from a Rhesus monkey gene is complementary to, or a fragment of, any portion of any of SEQ ID NOS. 18599-35840 or SEQ ID NOS. 36075-43225.

43. (canceled)

44. The nucleotide array of claim 41, wherein said polynucleotide probe from a Rhesus monkey gene is any of SEQ ID NOS 35841-36074.

45-52. (canceled)

53. The nucleotide array of claim 7, wherein said nucleotide array additionally comprises at least one polynucleotide probe complementary to, or a fragment of, any portion of any Rhesus monkey gene.

54. The nucleotide array of claim 53, wherein said polynucleotide probe from a Rhesus monkey gene is complementary to, or a fragment of, any portion of any of SEQ ID NOS. 18599-35840 or SEQ ID NOS. 36075-43225.

55. (canceled)

56. The nucleotide array of claim 53, wherein said polynucleotide probe from a Rhesus monkey gene is any of SEQ ID NOS 35841-36074.

57. The nucleotide array of claim 1, wherein said nucleotide array additionally comprises at least one polynucleotide probe complementary to, or a fragment of, any portion of a Rhesus monkey gene and at least one polynucleotide probe complementary to, or a fragment of, any portion of any human gene.

58. The nucleotide array of claim 2, wherein said nucleotide array additionally comprises at least one polynucleotide probe complementary to, or a fragment of, any portion of a Rhesus monkey gene and at least one polynucleotide probe complementary to, or a fragment of, any portion of any human gene.

59-64. (canceled)

65. The nucleotide array of claim 3, wherein said nucleotide array additionally comprises at least one polynucleotide probe complementary to, or a fragment of, any portion of a Rhesus monkey gene and at least one polynucleotide probe complementary to, or a fragment of, any portion of any human gene.

66-71. (canceled)

72. The nucleotide array of claim 4, wherein said nucleotide array additionally comprises at least one polynucleotide probe complementary to, or a fragment of, any portion of a Rhesus monkey gene and at least one polynucleotide probe complementary to, or a fragment of, any portion of any human gene.

73. The nucleotide array of claim 72, wherein said polynucleotide probe from a Rhesus monkey gene is complementary to, or a fragment of, any portion of any of SEQ ID NOS. 18599-35840 or SEQ ID NOS. 36075-43225 and said polynucleotide probe from a human gene is complementary to, or a fragment of, any portion of any of SEQ ID NOS. 43450-48714.

74. The nucleotide array of claim 72, wherein said polynucleotide probe from a Rhesus monkey gene is complementary to, or a fragment of, any portion of any of SEQ ID NOS. 18599-35840 or SEQ ID NOS. 36075-43225 and said polynucleotide probe from a human gene is any of SEQ ID NOS. 43226-48714.

75-76. (canceled)

77. The nucleotide array of claim 72, wherein said polynucleotide probe from a Rhesus monkey gene is any of SEQ ID NOS. 35841-36074 and said polynucleotide probe from a human gene is complementary to, or a fragment of, any portion of any of SEQ ID NOS. 43450-48714.

78. The nucleotide array of claim 72, wherein said polynucleotide probe from a Rhesus monkey gene is complementary to, or a fragment of, any portion of any of SEQ ID NOS. 35841-36074 and said polynucleotide probe from a human gene is any of SEQ ID NOS. 43226-48714.

79-92. (canceled)

93. The nucleotide array of claim 7, wherein said nucleotide array additionally comprises at least one polynucleotide probe complementary to, or a fragment of, any portion of a Rhesus monkey gene and at least one polynucleotide probe complementary to, or a fragment of, any portion of any human gene.

94. The nucleotide array of claim 93, wherein said polynucleotide probe from a Rhesus monkey gene is complementary to, or a fragment of, any portion of any of SEQ ID NOS. 18599-35840 or SEQ ID NOS. 36075-43225 and said polynucleotide probe from a human gene is complementary to, or a fragment of, any portion of any of SEQ ID NOS. 43450-48714.

95. The nucleotide array of claim 93, wherein said polynucleotide probe from a Rhesus monkey gene is complementary to, or a fragment of, any portion of any of SEQ ID NOS. 18599-35840 or SEQ ID NOS. 36075-43225 and said polynucleotide probe from a human gene is any of SEQ ID NOS. 43226-48714.

96-97. (canceled)

98. The nucleotide array of claim 93, wherein said polynucleotide probe from a Rhesus monkey gene is any of SEQ ID NOS. 35841-36074 and said polynucleotide probe from a human gene is complementary to, or a fragment of, any portion of any of SEQ ID NOS. 43450-48714.

99. The nucleotide array of claim 93, wherein said polynucleotide probe from a Rhesus monkey gene is any of SEQ ID NOS. 35841-36074 and said polynucleotide probe from a human gene is any of SEQ ID NOS. 43226-48714.

100-193. (canceled)
